(19) United States
(12) Patent Application Publication
(10) Pub. No.: US 2025/0120912 A1
(43) Pub. Date: Apr. 17, 2025

(54) SYNERGISTIC NANOMEDICINE DELIVERING TOPOISOMERASE I TOXIN (SN-38) AND INHIBITORS OF POLYNUCLEOTIDE KINASE 3'-PHOSPHATASE (PNKP) FOR ENHANCED TREATMENT OF COLORECTAL CANCER

(71) Applicant: THE GOVERNORS OF THE UNIVERSITY OF ALBERTA, Edmonton (CA)

(72) Inventors: Afsaneh LAVASANIFAR, Edmonton (CA); Mohammad Reza VAKILI, Edmonton (CA); Sams Mohammad Anowar SADAT, Richmond (CA)

(21) Appl. No.: 18/691,738

(22) PCT Filed: Sep. 15, 2022
(86) PCT No.: PCT/CA2022/051374

§ 371 Date: Mar. 13, 2024

Related U.S. Application Data
(60) Provisional application No. 63/244,315, filed on Sep. 15, 2021.

(51) Int. Cl. A61K 9/107; A61K 31/437; A61K 31/4745; A61K 47/60; A61K 47/69; A61P 35/00

(52) U.S. Cl. CPC A61K 9/1075; A61K 31/437; A61K 31/4745; A61K 47/60; A61K 47/6907; A61P 35/00

(57) ABSTRACT

Synergistic nanomedicine delivering topoisomerase I toxin (SN-38) and inhibitors of polynucleotide kinase 3′-phosphatase (PNKP) for enhanced treatment of colorectal cancer

### CROSS REFERENCE TO RELATED APPLICATION

This application claims priority to United States Provisional Patent Application number U.S. 63/244,315, filed 15 Sep. 2021, the entire contents of which is hereby incorporated by reference.

### FIELD

The present disclosure relates generally to synergistic nanomedicine delivering topoisomerase I toxin (SN-38) and inhibitors of polynucleotide kinase 3′-phosphatase (PNKP) for enhanced treatment of colorectal cancer.

### BACKGROUND

Combination therapy is a common approach in treating different types of cancer. Drug combinations may be used to provide additive effects on heterogenous tumor populations leading to better control of tumor growth. Alternatively, drug combinations may be used to sensitize cancer cells to the effect of common chemotherapeutics and show synergistic anticancer effects. Application of nanomedicine for the precise and controlled spatial and temporal delivery of synergistic drug combinations to tumor tissue and cells and/or synergistic dose ratios can enhance the effect of common chemotherapeutics to a greater extent. An example nanomedicine developed for this purpose is the U.S. Food and Drug Administration (FDA) approved liposomal formulation of daunorubicin and cytarabine (VYXEOS, Jazz Pharmaceuticals, Inc.) for treating acute myeloid leukemia [1].

### SUMMARY

In one aspect there is provided a method of treating a subject having colorectal cancer, suspected of having of treating colorectal cancer, or at risk of developing of treating colorectal cancer, comprising: administering to said subject,
a therapeutically effective amount of A83B4C63, or a pharmaceutically acceptable salt, solvate, hydrate, or prodrug thereof, and
a therapeutically effective amount of SN-38, or a pharmaceutically acceptable salt, solvate, hydrate, or prodrug thereof.

In one example, the SN-38, or a pharmaceutically acceptable salt, solvate, hydrate, or prodrug thereof is administered before administration of the A83B4C63, or a pharmaceutically acceptable salt, solvate, hydrate, or prodrug thereof.

In one example, wherein the A83B4C63, or a pharmaceutically acceptable salt, solvate, hydrate, or prodrug thereof is administered before administration of the PM₁₆-SN-38, or a pharmaceutically acceptable salt, solvate, hydrate, or prodrug thereof.

In one example, wherein the SN-38, or a pharmaceutically acceptable salt, solvate, hydrate, or prodrug thereof is administered substantially concurrently with the A83B4C63, or a pharmaceutically acceptable salt, solvate, hydrate, or prodrug thereof.

In one example, wherein the subject is a human.

In one example, wherein the therapeutically effective amount of A83B4C63 is administered in a micelle.

In one example, wherein the A83B4C63-containing micelle comprises block copolymer mPEO-b-PBCL, PEO-b-PBCL, PEO-PCL, PEO-PDLA, and/or PEO-PLGA.

In one example, wherein the A83B4C63-containing micelle comprises block copolymer mPEO-b-PBCL.

In one example, wherein the PBCL block of the block copolymer in the A83B4C63-containing micelle has a degree of polymerization of between 2 and 50, preferably 10-30.

In one example, wherein the PBCL block of the block copolymer in the A83B4C63-containing micelle has a degree of polymerization of 26.

In one example, wherein the concentration of A83B4C63 in the A83-containing micelle is between 1 μM and 100 μM, preferably between 5 μM and 40 μM.

In one example, wherein the therapeutically effective amount of SN-38 is administered in a micelle.

In one example, wherein the SN-38-containing micelle comprises block copolymer mPEO-b-PBCL, PEO-b-PBCL, PEO-PCL, PEO-PDLA, and/or PEO-PLGA.

In one example, wherein the SN-38-containing micelle comprises block copolymer mPEO-B-PBCL.

In one example, wherein SN-38 in the SN-38-containing micelle is conjugated to a block copolymer.

In one example, wherein the PBCL block of the block copolymer in the SN-38-containing micelle has a degree of polymerization of between 1 and 30, preferably between 5 and 25.

In one example, wherein the PBCL block of the block copolymer in the SN-38-containing micelle has a degree of polymerization of 16.

In one example, wherein the concentration of SN-38 in the micelle is between 0.001 μM and 10 μM, preferably between 0.005 μM and 1 μM.

In one example, wherein the therapeutically effective amount of A83B4C63 or SN-38 is administered in a composition.

In one example, wherein the therapeutically effective amount of A83B4C63 or SN-38 is administered in a pharmaceutical composition further comprising a pharmaceutically acceptable excipient, diluent, or carrier.

In one aspect there is provided a use of a therapeutically effective amount of A83B4C63, or a pharmaceutically acceptable salt, solvate, hydrate, or prodrug thereof, and a therapeutically effective amount of SN-38, or a pharmaceutically acceptable salt, solvate, hydrate, or prodrug thereof, for treating a subject having colorectal cancer, suspected of having of treating colorectal cancer, or at risk of developing of treating colorectal cancer.

In one aspect there is provided a use of a therapeutically effective amount of A83B4C63, or a pharmaceutically acceptable salt, solvate, hydrate, or prodrug thereof, and a therapeutically effective amount of SN-38, or a pharmaceutically acceptable salt, solvate, hydrate, or prodrug thereof, in the manufacture of a medicament for treating a subject having colorectal cancer, suspected of having of treating colorectal cancer, or at risk of developing of treating colorectal cancer.

In one example, wherein the SN-38, or a pharmaceutically acceptable salt, solvate, hydrate, or prodrug thereof is for administration before administration of the A83B4C63, or a pharmaceutically acceptable salt, solvate, hydrate, or prodrug thereof.

In one example, wherein the A83B4C63, or a pharmaceutically acceptable salt, solvate, hydrate, or prodrug thereof is for administration before administration of the SN-38, or a pharmaceutically acceptable salt, solvate, hydrate, or prodrug thereof.

In one example, wherein the SN-38, or a pharmaceutically acceptable salt, solvate, hydrate, or prodrug thereof is for administration substantially concurrently with the A83, or a pharmaceutically acceptable salt, solvate, hydrate, or prodrug thereof.

In one example, wherein the subject is a human.

In one aspect there is provided a micelle composition comprising SN-38 and A83B4C63.

In one example, wherein the micelle comprises block copolymer mPEO-b-PBCL, PEO-b-PBCL, PEO-PCL, PEO-PDLA, and/or PEO-PLGA.

In one example, wherein the micelle comprises block copolymer mPEO-b-PBCL.

In one example, wherein SN-38 is conjugated to a block copolymer.

In one example, wherein the PBCL block of the block copolymer has a degree of polymerization between 1 and 50.

In one example, wherein the concentration of A83B4C63 is between 1 μM and 100 μM, preferably between 5 μM and 40 μM, and the concentration of SN-38 is between is between 0.001 μM and 10 μM, preferably between 0.005 μM and 1 μM.

In one aspect there is provided a composition comprising SN-38 and A83B4C63.

In one aspect there is provided a pharmaceutical composition comprising SN-38 and A83B4C63, a pharmaceutically acceptable excipient, diluent, or carrier.

In one aspect there is provided a method of treating colorectal cancer in a subject with of treating colorectal cancer, suspected of having of treating colorectal cancer, or at risk of developing of treating colorectal cancer, comprising: administering to said subject a therapeutically effective amount a micelle composition of claims 27 to 32, a composition of claim 33, or a pharmaceutical composition of claim 34.

In one example, wherein the subject is a human.

In one aspect there is provided a use of a therapeutically effective amount of a micelle composition of claims 27 to 32, a composition of claim 33, or a pharmaceutical composition of claim 34, or a pharmaceutically acceptable salt, solvate, hydrate, or prodrug thereof, for treating a subject having colorectal cancer, suspected of having of treating colorectal cancer, or at risk of developing of treating colorectal cancer.

In one aspect there is provided a use of a therapeutically effective amount of a micelle composition of claims 27 to 32, a composition of claim 33, or a pharmaceutical composition of claim 34, or a pharmaceutically acceptable salt, solvate, hydrate, or prodrug thereof, in the manufacture of a medicament for treating a subject having colorectal cancer, suspected of having of treating colorectal cancer, or at risk of developing of treating colorectal cancer.

In one example, wherein the subject is a human.

### BRIEF DESCRIPTION OF THE FIGURES

Embodiments of the present disclosure will now be described, by way of example only, with reference to the attached Figures.

FIG. 1: Scheme for (A) the synthesis of mPEO₁₁₄-b-PBCL₁₆/SN-38; (B) Self-assembly to mixed micelles composed of mPEO₁₁₄-b-PBCL₁₆/SN-38 and mPEO₁₁₄-b-PBCL₂₆ co-encapsulating A83B4C63. PM₁₆-SN-38 is the abbreviation used for mPEO₁₁₄-b-PBCL₁₆/SN-38, while PM₂₆/A83 is the abbreviation used for mPEO₁₁₄-PBCL₂₆ micelles physically encapsulating A83B4C63. A mixture of the two polymer co-encapsulating SN-38 and A83B4C63 is abbreviated as PM₁₆-SN-38: PM₂₆/A83.

FIG. 2: TEM images of the polymeric micelles formed from (A) PM₁₆-SN-38, (B) PM₁₆-SN-38: PM₂₆, (C) PM₁₆-SN-38: PM₂₆/A83, (D) PM₂₆/A83, and (E) PM₂₆. Images were obtained at a magnification of 110,000× at 75 kV. The bar in the bottom left corner of each image indicates a scale of 100 nm. Hydrodynamic diameter (Dn), PDI, and size distribution of (F) PM₁₆-SN-38, (G) PM₁₆-SN-38: PM₂₆, (H) PM₁₆-SN-38: PM₂₆/A83, (I) PM₂₆/A83, and (J) PM₂₆ micelles in aqueous medium were obtained by dynamic light scattering (DLS) using Zetasizer Nano (Malvern).

FIG. 3: Average (A) percentage of intensity and (B) PDI of PM₁₆-SN-38, PM₁₆-SN-38: PM₂₆, PM₂₆/A83, and PM₁₆-SN-38: PM₂₆/A83 micellar formulations (3 mg/mL) in the presence of SDS (20 mg/mL) at a ratio of 2:1 (v/v) as a function of time up to 24 h.

FIG. 4: (A) The A83B4C63 release profile from PM₂₆/A83 and PM₁₆-SN-38: PM₂₆/A83 micelles compared to the free A83 from dialysis tubing (MWCO=3.5 kDa) in aqueous solution (4% albumin in ultrapure water) at 37° C. (B) The SN-38 release profile from PM₁₆-SN-38, PM₁₆-SN-38: PM₂₆, and PM₁₆-SN-38: PM₂₆/A83 micelles compared to the free SN-38 from dialysis tubing (MWCO=3.5 kDa) in aqueous solution (4% albumin in ultrapure water) at 37° C. Significances of the differences were considered if *p≤0.05, **p≤0.01, ***p≤0.001, ****p≤0.0001 following one-way ANOVA multiple comparison test following Tukey's method. Data are represented as mean±SD (n=3).

FIG. 5: Analysis of additive versus synergistic effects for the combination of TOP1 and PNKP inhibitors as free or part of different formulations at indicated concentrations in (A, C, and E) HCT116 and (B, D, and F) HT-29 cells. Data is a representative model of three independent experiments. Areas of highest synergy are found within the bordered regions marked in C-F.

FIG. 6: In vitro cytotoxicity of free irinotecan, irinotecan+PM₂₆/A83, SN-38, SN-38+PM₂₆/A83, PM₁₆-SN-38, and PM₁₆-SN-38+PM₂₆/A83 in (A, C, and E) HCT116 and (B, D, and F) HT-29 cell lines after 24 h, 48 h, and 72 h incubation at 37° C. in 5% CO₂. The cells were treated with the free TOP1 inhibitor or their polymeric micelles at a range of concentration from 0.001 to 100 μM. SN-38 as free drug was solubilized with the aid of DMSO and the untreated cells received only 0.1% DMSO. The concentration of A83B4C63 was 10 μM. Each point represents mean±SD (n=4).

FIG. 7: In vitro cytotoxicity of PM₁₆-SN-38: PM₂₆ (black) and PM₁₆-SN-38: PM₂₆/A83 (gray) in (A, C, and E) HCT116 and (B, D, and F) HT-29 cell lines after 24 h, 48 h, and 72 h incubation at 37° C. in 5% CO₂. The cells were treated at a range of concentration from 0.001 to 100 μM of SN-38. The concentration of A83B4C63 was 10 μM. Each point represents mean±SD (n=4).

FIG. 8: Western blot detection of (A and B) cleaved PARP, (E and F) γ-H2AX, (I and J) cleaved caspase-7, and (M and N) cleaved caspase-3 in both cell lines after 6 h exposure to the respective treatments at 37° C. in 5% CO₂ (n=3). β-actin was used as a loading control. The conditions for all sample preparations and western blots were the same. Data are expressed as mean±SD (n=3). The statistical analysis of (C and D) cleaved PARP, (G and H) γ-H2AX, (K and L) cleaved caspase-7, and (O and P) cleaved caspase-3 was performed after normalized to β-actin. Differences were considered significant if * P≤0.05, ** P≤0.01, *** P≤0.001, and **** P≤0.0001 following two-way ANOVA followed by Tukey's method.

### DETAILED DESCRIPTION

The present disclosure relates generally to synergistic nanomedicine delivering topoisomerase I toxin (SN-38) and inhibitors of polynucleotide kinase 3′-phosphatase (PNKP) for enhanced treatment of colorectal cancer.

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As described herein, in one aspect, there is described compounds, compositions, methods and kits for treating a subject suspect of having colorectal cancer.

In one aspect, there is provided A method of treating a subject having colorectal cancer, suspected of having of treating colorectal cancer, or at risk of developing of treating colorectal cancer, comprising: administering to said subject, —a therapeutically effective amount of A83B4C63, or a pharmaceutically acceptable salt, solvate, hydrate, or prodrug thereof, and—a therapeutically effective amount of SN-38, or a pharmaceutically acceptable salt, solvate, hydrate, or prodrug thereof.

The term “subject”, as used herein, refers to any human or non-human animal. Non-limiting examples of a subject include humans, non-human mammal, primates, rodents, companion animals (including but not limited to dogs, cats, mice, rats), livestock (including but not limited to horses, sheep, cattle, pigs), reptiles, amphibians, and the like. A subject may also be referred to as a “patient”.

In a specific example, the subject is a human.

The terms “cell proliferative disorder” and “proliferative disorder” refer to disorders that are associated with some degree of abnormal cell proliferation. In one embodiment, the cell proliferative disorder is cancer.

The term “cancer” refers to or describes the physiological conditions in a subject generally characterized by inappropriate cellular proliferation, abnormal or excessive cellular proliferation. Cancers may be solid or non-solid cancers. Cancers may be a primary cancer and/or metastatic cancer. Cancers include, but are not limited to, a solid cancer, a non-solid cancer, a primary cancer, or a metastatic cancer.

The term “colorectal cancer” refers to any cancer of the large bowel, which includes the colon (the large intestine from the cecum to the rectum), the rectum, and/or the appendix, either in vivo or in vitro, and encompasses cell lines derived from colorectal cancer cells.

The most common colorectal cancer (CRC) cell type is adenocarcinoma that accounts for about 95% of cases. Other types of CRC include inter alia lymphoma and squamous cell carcinoma.

In some examples, the colorectal cancer is an adenocarcinoma.

In some examples, the colorectal cancer is colon adenocarcinoma (COAD).

In some examples, the colorectal cancer is rectal cancer, such as rectal adenocarcinoma (READ).

As used herein “a subject with colorectal cancer” refers to any subject, in particular human, having developed atypical and/or malignant cells in the lining and/or the epithelium of the large intestine, rectum and. or appendix. This includes CRC patients independent of the stage and form of the CRC. Patients suffering from, colorectal cancer also include patients which are recurrent with colorectal cancer, i.e. patients wherein after surgical treatment the tumor could no longer be detected for a certain time span, but wherein the cancer has returned in the same or different part of the large intestine, rectum and/or appendix and/or wherein metastases have developed at different sites of the patient's body such as in the liver, lung, peritoneum, lymph nodes, brain and/or bones. In another example, a patient suffering from CRC is a patient wherein the initial tumor has already been treated surgically and the CRC is non-metastatic.

In some non limiting examples, CRC may be staged according to the Dukes system, the Astier-Colier system or the TNM system (tumors/nodes/metastases).

The TNM system of the American Joint Committee of Cancer (AJCC) describes the size of the primary tumor (T), the degree of lymph node involvement (N) and whether the cancer has already formed distant metastasis (M), i.e. spread to other parts of the body. Here, stages 0, IA, IB, 11 A, I I B, III and IV are defined based on the determined T-, N- and M-values. A corresponding staging scheme can be derived from the Cancer Staging Manual of the AJCC. Another system for staging of colorectal cancer is the Dukes system established by the British pathologist Cuthbert Dukes, defining cancer stages A, B, C and D. This system was adapted by Astler and Coiier, who further subdivided stages B and C (“modified Astler-Coller classification”).

As used herein, CRC patient includes patients staged according to any staging system used and irrespective of the stage diagnosed.

In some examples of the methods and the use described herein, the patient is suffering from metastatic colorectal cancer. Metastatic colorectal cancer includes any form of CRC wherein the cancer has spread from its original starting point to another part of the body such as the lymph nodes or any other organs. In particular, patients suffering from metastatic colorectal cancer include patients wherein the N value as defined in the TNM system is NI (metastasis in 1 to 3 regional lymph nodes), N 1 a (metastasis in 1 regional lymph node), N i b (metastasis in 2-3 regional lymph nodes), N I c (tumor deposit(s) in the sub serosa, mesentery, or non-peritonealized pericolic or perirectal tissues without regional nodal metastasis), N2 (metastasis in 4 or more regional lymph nodes), N2a (metastasis in 4 to 6 regional lymph nodes) or N2b (metastasis in 7 or more regional lymph nodes) and/or the M value according to the TNM system is MI (distant metastasis), Mia (metastasis confined to one organ or site (e.g. liver, lung, ovary, non-regional node)) or M I b (metastasis in more than one organ/site or the peritoneum). This includes patients in stage III A, III B, IIIC, IVA and IVB according to the TNM system, in stage C according to the Dukes system and in stages C 1, C2 and/or C3 according to the Astler-Coller system.

In some examples, different (histopathologic) forms of CRC include adenocarcinoma in situ, adenocarcinoma, medullary carcinoma, mucionous carcinoma (colloid type), signet ring cell carcinoma, squamous cell (epidermoid) carcinoma, adenosquamous carcinoma, small cell carcinoma, undifferentiated carcinoma and/or carcinoma NOS (not otherwise specified). Histological grades include GX (grade cannot be assessed), GI (well differentiated), G2 (moderately differentiated), G3 (poorly differentiated) and G4 (undifferentiated).

By the terms “treating” or “lessening the severity”, it is to be understood that any reduction using the methods, compounds and composition disclosed herein, is to be considered encompassed by the invention. Treating or lessening in severity, may, in one embodiment comprise enhancement of survival, or in another embodiment, halting disease progression, or in another embodiment, delay in disease progression, or in another embodiment, diminishment of pain, or in another embodiment, delay in disease spread to alternate sites, organs or systems. It is to be understood that any clinically beneficial effect that arises from the methods, compounds and compositions disclosed herein, is to be considered to be encompassed by the invention.

In a specific example, treatment is carried out in vivo.

In a specific example, treatment is carried out in vitro, including but not limited to, in test tube, in cultured cells (both adherent cells and non-adherent cells), and the like.

In a specific example, treatment is carried out ex vivo, including but not limited to, in test tube, in cultured cells (both adherent cells and non-adherent cells), and the like.

The term “prognosis” as used herein refers to the prediction of the likelihood of cancer-attributable death or progression, including recurrence, metastatic spread, and drug resistance, of a neoplastic disease.

The term “pharmaceutically effective amount” as used herein refers to the amount of a drug or pharmaceutical agent that will elicit the biological or medical response of a tissue, system, animal or human that is being sought by a researcher or clinician. This amount can be a therapeutically effective amount.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Thus, the term “carrier” or “excipient” may refer to a non-toxic solid, semi-solid or liquid filler, diluent. The term includes solvents, dispersion, media, coatings, isotonic agents, and adsorption delaying agents, and the like. The use of such media and agents for pharmaceutically active substances is well known in the art.

As used herein, the term “pharmaceutically-acceptable salts” refers to the conventional nontoxic salts or quaternary ammonium salt. These salts can be prepared in situ in the administration vehicle or the dosage form manufacturing process, or by separately reacting a compound in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

A “pharmaceutical composition” as used herein refers to a chemical or biological composition suitable for administration to a mammalian subject. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

The term “functional derivative” as used herein refers to a molecule that retains a biological activity (either function or structural) that is substantially similar to that of the original compound. A functional derivative or equivalent may be a natural derivative or is prepared synthetically. For example, the term “derivative”” as used herein may refer to a chemical substance related structurally to another, i.e., an “original” substance, which can be referred to as a “parent” compound. A “derivative” can be made from the structurally-related parent compound in one or more steps. The general physical and chemical properties of a derivative are also similar to the parent compound.

Also encompassed is prodrug or “physiologically functional derivative”. The term “physiologically functional derivative” as used herein refers to compounds which are not pharmaceutically active themselves but which are transformed into their pharmaceutically active form in vivo, i.e. in the subject to which the compound is administered.

As used herein, a “prodrug” refers to a compound that can be converted via some chemical or physiological process (e.g., enzymatic processes and metabolic hydrolysis). The term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound.

The term, as used herein, “micelle” refers to an organized auto assembly of molecules formed in a liquid where the hydrophilic regions are in contact with the surrounding solvent and the hydrophobic regions are sequestered in the center or core of the micelle. In some embodiments, the micelle may be a nanoparticle. In some examples, the micelle comprises block copolymers. In specific examples, the block copolymers are mPEO-b-PBCL, PEO-b-PBCL, PEO-PCL, PEO-PDLA, and/or PEO-PLGA. In further embodiments, each block within the copolymer can be synthesized with varying degrees of polymerization using methods well known in the art.

In some examples, compound(s) and/or composition(s) of the present application may be administered with a physiologically acceptable carrier. A physiologically acceptable carrier is a formulation to which the compound can be added to dissolve it or otherwise facilitate its administration. Non limiting examples include, but are not limited to, water, saline, physiologically buffered saline.

In some examples, compound(s) and/or composition(s) and pharmaceutically acceptable composition(s) as described herein can be administered by parenteral administration, in the form of aqueous or non-aqueous isotonic sterile injection solutions or suspensions. These solutions and suspensions can be prepared from sterile powders or granules having one or more of the carriers or diluents mentioned for use in the formulations for oral administration. Compound(s) and/or composition(s) as described herein can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art, as know by the skilled worker.

Liquid dosage forms for oral administration include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

It will be appreciated that the amount of active ingredient that can be combined with the carrier materials to produce a single dosage form varies depending upon the mammalian host treated and the particular mode of administration.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### Examples

### Abstract

Our research group has previously reported on the development of polymeric micelles (PM) s based on methoxy poly(ethylene oxide)-poly(α-benzyl carboxylate-e-caprolactone) (mPEO-b-PBCL) for delivery of novel inhibitors of a DNA repair enzyme, polynucleotide kinase 3′-phosphatase (PNKP). In this study, PMs co-encapsulating a novel inhibitor of PNKP, i.e., A83B4C63, and a potent topoisomerase I inhibitor, i.e., SN-38, were developed through physical encapsulation of A83B4C63 and chemical conjugation of SN-38 in mPEO-b-PBCL PMs. This improved the solubilized level of both compounds in aqueous media. The loading content (% w/w) of the chemically conjugated SN-38 and physically loaded A83B4C63 to mPEO-b-PBCL micelles was 7.91±0.66 and 16.13±0.11, respectively. Notably, the average diameter of PMs co-encapsulating both SN-38 and A83B4C63 was below 60 nm but still larger than PMs encapsulating either of these drugs alone. The release of A83B4C63 from PMs co-encapsulating both drugs, was 76.36±1.41% within 24 h, which was significantly higher than that of A83B4C63-encapsulated micelles (42.70±0.72). In contrast, the release of SN-38 from PMs co-encapsulating both drugs were 44.15±2.61% at 24 h, which was significantly lower than that of SN-38-conjugated PMs (74.16±3.65). Cytotoxicity evaluations by MTS assay as analyzed by the Combenefit® software suggested a clear synergy between A83B4C63 (at a concentration range of 10-40 μM) and SN-38 (at a concentration range of 0.001-1 μM) either as free or PM formulations. PMs co-encapsulating A83B4C63 and SN-38 at drug concentrations within this range (10 μM for A83B4C63 and 0.05-1 μM for SN-38) showed slightly less activity to A83B4C63 PMs+SN-38 PMs or that of A83B4C63 PMs+free SN-38 at the same molar concentration in both cell lines, reflecting slower release of SN-38 from the co-encapsulated PMs. Following the expression of □-HA2X, cleaved PARP, caspase 3 and caspase 7, revealed equal activity for a mixture of PMs encapsulating either drug or PMs co-encapsulating both compounds. The overall result from the study shows a synergy between PMs of SN-38 and A83B4C63 as a mixture of two PMs for individual drug or PMs co-encapsulating both drugs.

### 1. INTRODUCTION

Combination therapy is a common approach in treating different types of cancer. Drug combinations may be used to provide additive effects on heterogenous tumor populations leading to better control of tumor growth. Alternatively, drug combinations may be used to sensitize cancer cells to the effect of common chemotherapeutics and show synergistic anticancer effects. Application of nanomedicine for the precise and controlled spatial and temporal delivery of synergistic drug combinations to tumor tissue and cells and/or synergistic dose ratios can enhance the effect of common chemotherapeutics to a greater extent. An example nanomedicine developed for this purpose is the U.S. Food and Drug Administration (FDA) approved liposomal formulation of daunorubicin and cytarabine (VYXEOS, Jazz Pharmaceuticals, Inc.) for treating acute myeloid leukemia [1].

Our research group has previously reported on the development of novel inhibitors of a DNA repair enzyme, polynucleotide kinase 3′-phosphatase (PNKP), that were able to sensitize colorectal cancer (CRC) cells to the DNA damaging effect of topoisomerase I (TOP1) inhibitor, irinotecan, as well as ionizing radiation (IR), in vitro [2, 3]. A polymeric micelle (PM) formulation of a small molecule inhibitor of PNKP, known as A83B4C63, was shown to redirect the encapsulated A83B4C63 away from normal tissue to HCT116 tumor xenograft. As a result, the PM formulation of A83B4C63, which was made from self-assembly of methoxy poly(ethylene oxide)-b-poly(α-benzyl carboxylate-e-caprolatcone) (mPEO-b-PBCL) was effective in increasing the inhibitory effect of IR on the growth of HCT116 xenografts, a CRC tumor, in vivo.

Irinotecan, also known as CPT-11, Camptosar, 7-ethyl-10-[4-(1-piperidino)-1-piperidino] carbonyloxycamptothecin, is a semi-synthetic camptothecin derivative, approved by the FDA for primary CRC treatment [4]. Irinotecan, is the water-soluble prodrug of SN-38, and is metabolized by endogenous carboxylesterase into its biologically active metabolite; 7-ethyl-10-hydroxycamptothecin or SN-38. The active metabolite of irinotecan is 1,000 times more potent than its prodrug [5]. Besides, the conversion from irinotecan to SN-38 is very limited and slow (<10%) and cell dependent. [6, 7]. Nanodelivery of SN-38 and its combination with inhibitors of PNKP was hypothesized to overcome the problem of poor-solubility for SN-38 and at the same time enhance the potency of anti-TOP1 treatment in CRC.

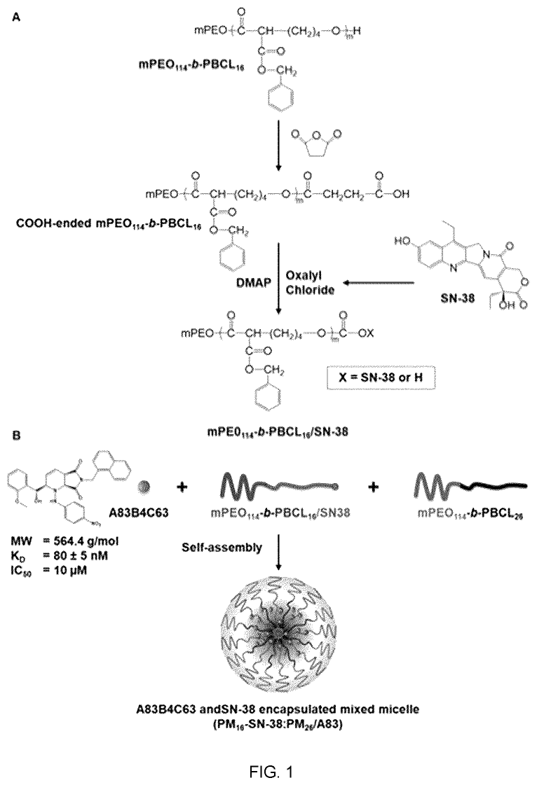

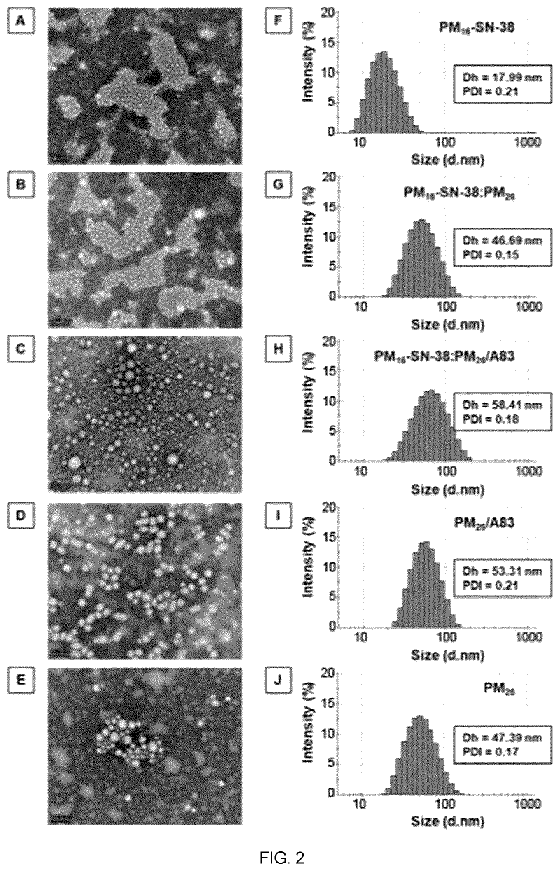

In the current manuscript, we described development of PMs based on mPEO-b-PBCL for co-delivery of A83B4C63 and SN-38, a potent TOPI inhibitor, at synergistic ratios (FIG. 1) and characterized the physicochemical properties of this formulation making comparison with PMs for individual A83B4C63 or PMs for SN-38 alone. The effect of mixing the PMs of A83B4C63 as a separate entity to that of TOPI inhibitor as free or PM formulation or upon co-encapsulation in the same PM, on their anticancer activity in two compounds in CRC cells have been explored.

### 2. MATERIALS AND METHODS

### 2.1. Materials

Methoxy-polyethylene oxide (mPEO) (average molecular weight of 5000 gmol⁻¹), sodium dodecyl sulfate (SDS), and bovine serum albumin (BSA) and all research grade organic solvents were purchased from Sigma (St. Louis, MO, USA). α-benzyl carboxylate-ε-caprolactone monomer was synthesized by Alberta Research Chemicals Inc. (Edmonton, AB, Canada). Stannous octoate was purchased from MP Biomedicals Inc. (Tuttlingen, Germany). (S)-4, 11-Diethyl-4, 9-di-OH-1, 12-dihydro-4H-2-oxa-6,12α-diaza-dibenzo[b, h]fluorene-3,13-dione (SN-38) (purity>97%) was purchased from abcr GmbH (Karlsruhe, Germany). All other chemicals and reagents used were of analytical grade.

### 2.2. Synthesis of Block Copolymers and A83B4C63

The block copolymers of methoxy poly(ethylene oxide)-b-poly(α-benzyl carboxylate-ε-caprolactone (mPEO-b-PBCL) with two different degrees of polymerization (DP=16 and 26) for the PBCL block were synthesized by ring-opening polymerization of α-benzyl carboxylate-ε-caprolactone using methoxy-PEO (MW: 5000 gmol⁻¹) as an initiator and stannous octoate as catalyst according to a previously described method [9]. The polysubstituted imidopiperidine compound, 2-[hydroxy (2-methoxyphenyl)methyl]-6-(naphthalene-1-ylmethyl)-1-[(4-nitrophenyl)amino]-2H, 4aH, 7aH-pyrrolo[3,4-b]pyridine-5,7-dione or A83B4C63 was synthesized using a three-component aza [4+2]/allylboration reaction and purified to homogeneity via HPLC as previously described method [10]. The structure of the compound was confirmed by 1H NMR, IR, and LC-MS as previously reported [2].

### 2.3. Synthesis of Carboxyl-Terminated mPEO114-b-PBCL16 Block Copolymers

The mPEO-b-PBCL block copolymer with DP of 16 (mPEO₁₁₄-b-PBCL₁₆) was chemically modified through reaction with succinic anhydride to generate mPEO-b-PBCL copolymer with the end-capped carboxylic acid functional group (mPEO₁₁₄-b-PBCL₁₆-COOH) as previously described with minor modifications [11]. In brief, 4 g of mPEO₁₁₄-b-PBCL₁₆ and succinic anhydride (1.5 times molar excess of polymer) were mixed and placed in a previously flame-dried ampoule. The ampoule was sealed and kept in an oven for 6 h reaction at 140° C. Thereafter, COOH-terminated block copolymers were dissolved in dichloromethane and subsequently, precipitated in hexane and centrifuged at 3000 rpm [12]. The supernatant was discarded, and the final product was washed twice with hexane, then dried in a vacuum oven overnight at room temperature.

### 2.4. Conjugation of SN-38 to mPEO114-b-PBCL16-COOH Copolymers

Conjugation of SN-38 to mPEO₁₁₄-b-PBCL₁₆-COOH was performed by activation of carboxylic acid terminal group on PBCL block using oxalyl chloride and 4-dimethylaminopyridine (DMAP). At first, 1 g of mPEO₁₁₄-b-PBCL₁₆-COOH was added into 5 mL of refluxed dichloromethane (DCM) in a round bottom flask under continuous magnetic stirring and heat. Then, 200 μL of oxalyl chloride was added to the reaction mixture under the same reflux condition and left for 5 h. During the reaction, the acidity of the reaction mixture was checked every hour by pH paper. After 5 h of the reaction, the content was semi-dried by heat. When the reaction content reached to ¼ of the original volume, dry anhydrous hexane (˜ 5 mL) was added into the semi-dried polymer in the flask. Immediately after discarding the hexane, residual dried polymer was collected inside the flask. Meanwhile, 50 mg of SN-38 and 30 mg of DMAP were added in 5 mL of dimethylformamide (DMF), subsequently vortexed, and water bath sonicated until SN-38 was completely dissolved. Then, the solution of SN-38 and DMAP was transferred to round bottom flask which contained the polymer. This flask was then placed in ice-water bath while stirring under Ar gas. After 20 min, the ice was removed, and the reaction was left under continuous stirring at room temperature overnight. The next day, the reaction mixture was identified with reddish color and was diluted with 7 mL of dimethyl sulfoxide (DMSO), then dialyzed against DMSO for 48 h and deionized water for 24 h to remove unreacted SN-38 and other impurities. The solution inside the dialysis bag was freeze-dried to obtain a dry product.

### 2.5. Characterization of Block Copolymers and Drug-Copolymer Conjugates

The synthesized mPEO-b-PBCL block copolymers and mPEO-b-PBCL-SN-38 conjugates were characterized for their number average molecular weights by 1H NMR (600 MHz Avance III—Bruker, East Milton, ON, Canada) using deuterated chloroform (CDCl₃) as solvent. The DP of PBCL block was calculated from the 1H NMR based on the ratio of the peak intensity of protons from the ethylene (—CH₂CH₂O—) moiety of PEO (δ=3.65 ppm) to the peak intensity of the protons from the (—OCH₂—) methylene of caprolactone block (δ=4.05 ppm), considering a molecular weight of 5000 gmol⁻¹ for the PEO block. The level of carboxylic acid termination of mPEO-b-PBCL block copolymer was also measured based on 1H NMR. The level of SN-38 conjugation was expressed in loading percentage (% w/w) with respect to the carboxylic acid-terminated residue of mEPO-b-PBCL.

### 2.6. Preparation of Polymer Micellar Formulations of A83B4C63 and/or SN-38 and their Characterization

Table 1 shows the formulation details of different samples under this study, including the mixing ratio between the PM components, i.e., mPEO₁₁₄-b-PBCL₁₆-SN-38, mPEO₁₁₄-b-PBCL₂₆, and/or A83B4C63, in each formulation. Nano-formulations were prepared as described before [2]. In brief, the weighted amounts of different PM components (with respective ratios according to Table 1) were dissolved in 1 mL of acetone. Then, this solution was added dropwise to 3 mL of aqueous phase (deionized water) and left overnight with continuous stirring with a magnetic bar under fume hood to completely evaporate the organic solvent. The un-encapsulated drug was removed by centrifugation at 11600×g for 5 min to obtain drug-loaded PMs. The size (Z-average diameter), polydispersity index (PDI), zeta potential (ZP), and critical micellar concentration (CMC) of the formulations prepared from individual or mixed block copolymer components were measured by dynamic light scattering (DLS) using a Malvern Zetasizer 3000 (Malvern Instruments Ltd, Malvern, UK). SN-38 conjugation level was measured using Synergy-H1 BioTEK microplate reader (Winooski, VT, USA) at a wavelength of 383 nm. A83B4C63 loading was measured using a Varian Prostar 210 HPLC system. Reversed phase chromatography was carried out with a Microsorb-MV 5 μm C18-100 Å column (4.6 mm×250 mm) with 20 μL of sample injected and eluted under isocratic conditions with a solution of 0.1% trifluroacetic acid: acetonitrile (1:1 v/V) at a flow rate of 0.7 mL/min at room temperature. Detection was performed at 280 nm wavelength for A83B4C63 using a Varian 335 Photodiode Array HPLC detector (Varian Inc., Palo Alto, CA, USA). In this study, A83B4C63 control was solubilized in DMSO for all in vitro experiments.

To investigate micellar stability, CMC of the block copolymers was also determined by DLS technique following a previously published method [13]. In brief, a series of micellar solutions of PM₂₆, PM₂₆/A83, PM₁₆-SN-38, PM₁₆-SN-38: PM₂₆, and PM₁₆-SN-38: PM₂₆/A83 with a polymeric concentration range of 1500 to 0.05 μg/mL were prepared in glass vials. The intensity of the scattered light for prepared samples was detected at an angle of 173° under single attenuator index. Measurements were carried out in polystyrene cells at 25° C. The count rate (Kcps) as a function of the intensity of the scattered light was plotted against the concentrations of block copolymers with or without payloads.

To determine the stability of micelles against dissociation, kinetic stability was also measured by DLS method as previously described [14]. In brief, micelles were prepared using individual copolymers or a combination of polymers with the compositions detailed in Table 1. The total polymer concentration was 3 mg/mL. Micelles were incubated with an aqueous solution of destabilizing agent, sodium dodecyl sulfate (SDS) (20 mg/mL) at a ratio of 2:1 (v/v). The intensity of scattered light and PDI were measured at different incubation time intervals (0, 1, 2, 4, 8, and 24 h).

TABLE 1
Formulation details for samples under study, including
the mixing ratio between the micellar components. The
number shown in the subscript of the formulation names indicates
the degree of polymerization of each block in the copolymers
as determined by ¹H NMR spectroscopy

| | | Mixing ratio |
|---|---|---|
| Formulation keys | Formulations details | (w/w) |
| PM [symbol] -8N-38 | mPEO [symbol] -b-PBCL [symbol] -SN-35 | — |
| PM [symbol] -SN-38:PM [symbol] | mPEO [symbol] -b-PBCL [symbol] -SN-35 | 1:1 |
| | — | |
| | mPEO [symbol] -b-PBCL [symbol] | |
| PM [symbol] -SN-38:PM [symbol] A83 | mPEO [symbol] -b-PBCL [symbol] -SN-35 | 1:1:0.33 |
| | — | |
| | mPEO [symbol] -b-PBCL [symbol] | |
| | — | |
| | A83B4C63 | |
| PM [symbol] A83 | mPEO [symbol] -b-PBCL [symbol] | 1:0.33 |
| | — | |
| | A83B4C63 | |
| PM [symbol] | mPEO [symbol] -b-PBCL [symbol] | — |
| A83 | A83B4C63 | — |
| | — | |
| | DMSO | |

[symbol]

indicates data missing or illegible when filed

### 2.7. Transmission Electron Microscopy (TEM)

The morphology of self-assembled structures under study was investigated by TEM using a Morgagni TEM (Field Emission Inc., Hillsboro, OR) with Gatan digital camera (Gatan, Pleasanton, CA). In brief, 20 μL of micellar solution with a polymer concentration of 0.5 mg/mL was placed on a copper-coated grid. The grid was held horizontally for 1 min to allow the colloidal particles to settle down. The excess fluid was removed by filter paper. The copper-coated grids holding the aqueous samples were then negatively stained by 2% phosphotungstic acid. After 2 min, the excess fluid was removed by filter paper and the grid was loaded into the TEM for image analysis.

### 2.8. In Vitro Drug Release

In vitro release of SN-38 and A83B4C63 from the self-assembled structures was investigated using dialysis-bag diffusion technique. Each dialysis bag (Spectra Por dialysis tubing, MWCO=3.5 kDa, Spectrum Laboratories, Rancho Dominguez, CA, U.S.A.), containing 3 mL of the micellar formulation in water or free SN-38 and A83B4C63 dissolved in DMSO, was immersed into 300 mL release medium (4% albumin in ultrapure water) maintained at 37° C. in a shaking water bath with 65 rpm (Julabo SW 22, Seelbach, Germany). At selected time intervals (0, 1, 2, 4, 6, 8, and 24 h), 300 μL aliquots from inside of the dialysis bag were withdrawn and replaced with equal volume of fresh release media (water). The concentrations of SN-38 in collected samples were determined by UV-Vis spectrophotometer (BioTEK, Winooski, VT, USA). Detection was performed at a wavelength of 383 nm. The concentrations of A83B4C63 in collected samples were measured and analyzed using a Varian Prostar 210 HPLC system. All experiments were carried out in triplicate.

### 2.9. Cell Lines

Two CRC cell lines, HCT116 and HT-29 originally purchased from ATCC were used. The cells were cultured at 37° C. in 5% CO₂ in a humidified incubator in a 1:1 mixture of Dulbecco's modified Eagle medium and F12 (DMEM/F12) supplemented with 10% FBS, 50 U/mL penicillin, 50 mg/mL streptomycin, 2 mmol/L L-glutamine, 0.1 mmol/L nonessential amino acids, and 1 mmol/L sodium pyruvate. All culture supplements were purchased from GIBCO, Life Technologies Inc. (Burlington, ON, CA).

### 2.10. In Vitro Synergy and Antagonism Evaluation

The day before the treatment, 2×10³ HCT116 and HT-29 cells were plated in each well of 96-well flat-bottomed plates. Then cells were treated with irinotecan, PM₂₆/A83, SN-38, PM₁₆-SN-38, irinotecan+PM₂₆/A83, SN-38+PM₂₆/A83, PM₁₆-SN-38+PM₂₆/A83 for 48 h. The cells were treated with the formulations at 0.001, 0.01, 0.1, 1, 10, and 100 μM concentrations for irinotecan and SN-38 formulations. The treating concentrations of A83B4C63 as part of formulations were 5, 10, 20, and 40 μM for the combination treatments and the control cells received only 0.1% DMSO. After the incubation time point, cells were treated with the MTS reagent (CellTiter 96® AQueous One Solution Cell Proliferation Assay, Promega, USA) to measure their viability. Each experiment was performed in quadruplicates. The cell viability percentages were calculated using the following formula:

$$Cell viability ( \% ) = \frac{Absorbance of treated cells - Absorbance of blank well}{Absorbance of untreated cells \text{?} Absorbance of blank well} \times 100$$

$$\text{?} \text{indicates text missing or illegible when filed}$$

The cell viability data following a combination template was processed using Loewe classical synergy models in Combenefit® software program [15].

### 2.11. In Vitro Cytotoxicity Assay

The CellTiter 96® AQueous One Solution Cell Proliferation Assay (MTS) kit from Promega, USA was used to assay the cytotoxicity of irinotecan, irinotecan+PM₂₆/A83, SN-38, SN-38+PM₂₆/A83, PM₁₆-SN-38, PM₁₆-SN-38+PM₂₆/A83, PM₁₆-SN-38: PM₂₆, and PM₁₆-SN-38: PM₂₆/A83 in HCT116 and HT-29 cells according to the provided company protocol. In brief, 2×10³ cells were plated in each well of 96-well flat-bottomed plates 24 h prior to the treatments. Then, cells were treated with the formulations at the concentration range of 0.001 to 100 μM for irinotecan and SN-38 formulations. The PM₂₆/A83 with a fixed concentration as of 10 μM was used for the combination treatments and the control cells received only 0.1% DMSO. The choice of this concentration was based on the results of synergy evaluation, in which at a concentration of 10 μM for A83B4C63, a synergy with SN-38 at a concentration range of 0.01-1 μM has been observed. After different experimental incubation time points (24, 48, and 72 h), 20 μL of MTS reagent was added in each well and further incubated for 1 h at 37° C. before measuring the absorbance at 490 nm using BioTEK microplate reader. Each experiment was performed in quadruplicates. The cell viability percentages were calculated using the formula mentioned in previous method section.

### 2.12. Western Blot Analysis

Western blotting was performed to assess the in vitro levels of caspase-3, caspase-7, PARP, and γ-H2AX proteins after treating both HCT116 and HT-29 cell lines with A83, irinotecan, irinotecan+PM₂₆/A83, SN-38, SN-38+PM₂₆/A83, PM₁₆-SN-38, PM₁₆-SN-38+PM₂₆/A83, PM₁₆-SN-38: PM₂₆, PM₁₆-SN-38: PM₂₆/A8B4C63, and PM₂₆/A83. In brief, 1×10⁶ cells were plated in each well of 6-well plates 24 h prior to the treatments. Then, the cells were treated with the media containing various treatments the final concentrations equivalent to the respective relative IC₅₀ concentrations of irinotecan, SN-38, PM₁₆-SN-38, and PM₁₆-SN-38: PM₂₆ for 6 h in combination with or without 10 μM PM₂₆/A83. The cells of 3 controls received only 0.1% DMSO, 10 μM A83, and 10 μM PM₂₆/A83, respectively. Protein extracts for western blot analysis were prepared using commercial RIPA lysis buffer (ThermoFisher Scientific, Canada) supplemented with a cocktail of protease inhibitors (Millipore Sigma, Canada). Protein concentrations were measured using the BCA assay kit (Pierce/ThermoFisher Scientific, Canada) according to the manufacturer's protocol. Equal concentrations of protein were separated by SDS-PAGE and transferred to nitrocellulose membranes. After blocking with 5% skimmed milk in TBST (50 mM Tris-HCl, pH 7.4, 150 mM NaCl, and 0.1% Tween 20), the blots were incubated with the respective primary antibodies (cleaved caspase-3 cat #9661S, cleaved caspase-7 cat #9491S, PARP cat #9542S, phospho-histone H2A.X (Ser139) cat #9718S) and secondary antibody (HRP-linked anti-rabbit IgG cat #7074S) purchased from Cell Signaling Technology (Whitby, ON, Canada). The protein bands were detected using an enhanced chemiluminescence (ECL) based system (Pierce/ThermoFisher Scientific, Canada). The band intensities for the PNKP protein were quantified by performing optical density analysis using ImageJ software. Each experiment was carried out in triplicate.

### 2.13. Statistical Analysis

Data are shown as mean±standard error of at least three experiments. GraphPad Prism9 software (La Jolla, CA, USA) was used for statistical analysis. The significance of difference between groups was assessed using one-way ANOVA followed by Tukey's post-hoc analysis. If a significant difference was found among the groups, median ranks between pairs of groups were compared using the Mann-Whitney U test. Differences in the physicochemical characterization of micellar formulations were also tested using the unpaired student's t-test. A value of p≤0.05 was considered as statistically significant in all experiments.

### 3. RESULTS

### 3.1. Characterization of Block Copolymers and PM Formulations

The ¹H NMR spectra of PM₁₆-SN-38 and peak assignments are shown in Supplementary FIG. 1. Using 1H NMR data as reported before, the DP of PBCL block in mPEO-b-PBCL copolymers used for SN-38 conjugation or A83B4C63 loading was found to be 16 and 26, respectively [2, 11, 13, 16, 17]. The end-capped carboxylic acid functional group conversion of PBCL block of mPEO-b-PBCL copolymer was 65-67%. The conjugated content (% w/w) of SN-38 to mPEO-b-PBCL-COOH was 15.67±0.34 as measured by UV spectroscopy.

The physicochemical characterizations of the self-assembled PM₁₆-SN-38, PM₁₆-SN-38: PM₂₆, PM₁₆-SN-38: PM₂₆/A83, PM₂₆/A83, and PM₂₆ micelles were also performed by measuring their average diameter, surface charge, PDI, and CMC as summarized in Table 2. The average diameters of all the micellar formulations were below 100 nm with PDI range below 0.22. The average hydrodynamic diameter of PM₁₆-SN-38 was significantly lower (p<0.0001) than that of it mixed micelle with PM₂₆, i.e., PM₁₆-SN-38: PM₂₆ (without A83). Co-encapsulation of PM₁₆-SN-38 and A83B4C63 in PM₁₆-SN-38: PM₂₆/A83 resulted in larger size (58.41±0.29 nm) among all the formulations under study.

The mean zeta potential for PM₁₆-SN-38 was on the negative side. The zeta potential of PM₂₆/A83 and PM₂₆, on the other hand was neutral. When PM₁₆-SN-38 formed mixed micelles with PM₂₆ (without or with A83B4C63 encapsulation), i.e., in PM₁₆-SN-38: PM₂₆, PM₁₆-SN-38: PM₂₆/A83, the zeta potential shifted towards the neutral range (from 0.07 to 0.08 mV), which was significantly different from the zeta potential of PM₁₆-SN-38 micelles (p<0.003) but not different from that of PM₂₆ samples.

The measured CMCs of PM₁₆-SN-38, PM₁₆-SN-38: PM₂₆, PM₁₆-SN-38: PM₂₆/A83, PM₂₆/A83, and PM₂₆ were 0.33±0.09, 0.54±0.12, 0.47±0.22, 0.43+0.11, and 0.48±0.21 μg/mL, respectively (Table 2). No statistical significance was identified between the CMCs of the formulations under study.

The loading of SN-38 in the mixed micellar formulation, i.e., PM₁₆-SN-38: PM₂₆, PM₁₆-SN-38: PM₂₆/A83, of that in the PM₁₆-SN-38 owing to 1:1 molar mixing ratio of polymers. The A83B4C63 encapsulation, was found to significantly decrease in mixed micellar formulation of PM₁₆-SN-38: PM₂₆/A83 co-encapsulating both SN-38 and A83B4C63, compared to that of PM₂₆/A83 (p<0.05) (Table 2).

TABLE 2
Physicochemical characteristics of all self-assembled micellar formulations (n = 4).

| | | | | | | A83B4C63 |
|---|---|---|---|---|---|---|
| | | | Zeta Potential [symbol] ± | CMC [symbol] ± SD | SN-38 Loading [symbol] | Loading [symbol] |
| Micellar Formulations [symbol] | Size ± SD (rm) | PDI [symbol] ± SD | SD (mV) | (ug mL) | (% ww) | (% ww) |
| PM₁₆-SN-38 | 17.99 ± 0.04 [symbol] | 0.21 ± 0.01 | −1.69 ± 0.24 [symbol] | 0.33 ± 0.09 | 15.67 ± 0.34 [symbol] | — |
| PM₁₆-SN-38:PM26 | 46.69 ± 0.27 [symbol] | 0.15 ± 0.01 | 0.08 ± 0.02 | 0.54 ± 0.12 | 7.64 ± 0.83 | — |
| PM₁₆-SN-38:PM26/A83 | 55.41 ± 0.29 [symbol] | 0.18 ± 0.01 | 0.07 ± 0.01 | 0.47 ± 0.22 | 7.91 ± 0.66 | 16.13 ± 0.11 |
| PM₂₆/A83 | 53.31 ± 0.21 [symbol] | 0.19 ± 0.02 | 0.09 ± 0.01 | 0.46 ± 0.11 | — | 23.06 ± 0.63 [symbol] |
| PM₂₆ | 47.39 = 0.17 [symbol] | 0.17 ± 0.01 | 0.09 ± 0.06 | 0.48 ± 0.21 | — | — |

[symbol]

The number shown in the subscript indicates the degree of polymerization of each block as determined by [symbol] HNMR spectroscopy. [symbol]
Hydrodynamic diameter (Z average) determined by DLS. [symbol]
Average PDI of micellar size distribution. [symbol]
Average surface charge (zeta potential) of the micelles. [symbol]
Average CMC measured by DLS.
$\text{?} SN - 38 loading ( ww \% ) = \frac{Amount of conjugated SN - 38}{Total amount of polymer} \times 100 ; measured using UV - Vis spectrophotoscopy .$ $\text{?} A83B4C63 loading ( ww \% ) = \frac{Amount of conjugated SN - 38 or A83 B4 C63}{Total amount of polymer} \times 100 ; measured using HPLC .$ [symbol] Differences were considered significant at *p ≤ 0.05 (either one-way ANOVA multiple comparison test following Tukey's method or student's t-test) [symbol]
indicates data missing or illegible when filed

### 3.2. Transmission Electron Microscopy (TEM)

The morphology of the self-assembled structures was investigated by TEM, confirming the formation of spherical-shaped PM₁₆-SN-38, PM₁₆-SN-38: PM₂₆, PM₁₆-SN-38: PM₂₆/A83, PM₂₆/A83, and PM₂₆ micelles with uniform size (FIG. 2A-2E). Moreover, a similar distribution pattern in the micellar population having a clear boundary was observed in TEM images of all micelles, indicating the lower aggregation tendency of micelles. Dynamic light scattering profiles showed formulation of a single peak for micelles formed from one or a mixture of block copolymers understudy (FIG. 2F-2J). The trend of change in the average diameter and size distribution between samples was similar in the two methods of analysis, i.e., TEM and DLS. For instance, PM₁₆-SN-38 showed the smallest average diameter and narrow polydispersity in both methods (FIGS. 2A and 2F). Whereas PM₁₆-SN-38: PM₂₆/A83 showed larger average diameter and high polydispersity (FIGS. 2C and 2H).

### 3.3. Kinetic Stability

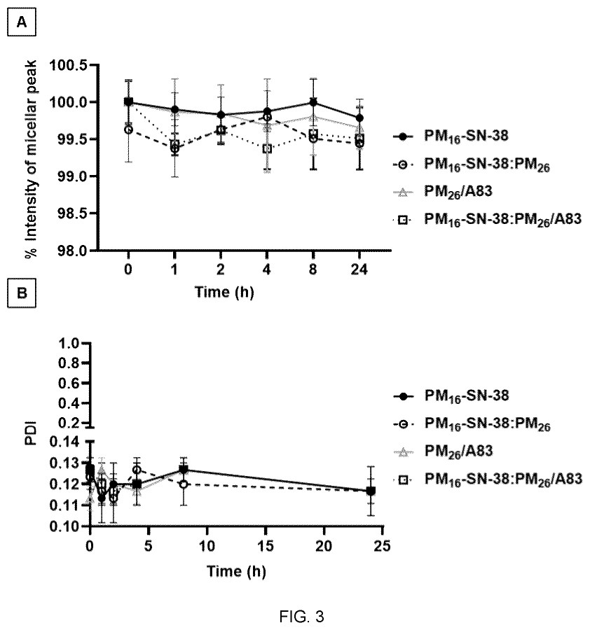

The kinetic stability of block copolymer micelles was investigated by following the dissociation of micellar peak in the presence of a destabilizing surfactant, i.e., SDS, after 0, 1, 2, 4, 8, and 24 h incubation using DLS technique. FIGS. 3A and 3B represent the % intensity of micellar peak and PDI, respectively, for PM₁₆-SN-38, PM₁₆-SN-38: PM₂₆, PM₁₆-SN-38: PM₂₆/A83, and PM₂₆/A83 micelles over 24 h in the presence of SDS. All the micellar formulations exhibited complete resistance against the destabilizing agent and remained intact throughout the incubation period up to 24 h. As shown in FIG. 3B, no significant dissociation of the micellar structures was detected since the DLS-obtained PDI was in similar ranges (0.1 to 0.14) for the formulations in this study following incubation with SDS. Overall data suggest that all the micellar formulations are kinetically stable regardless of the payloads and their composition.

### 3.4. In Vitro Drug Release

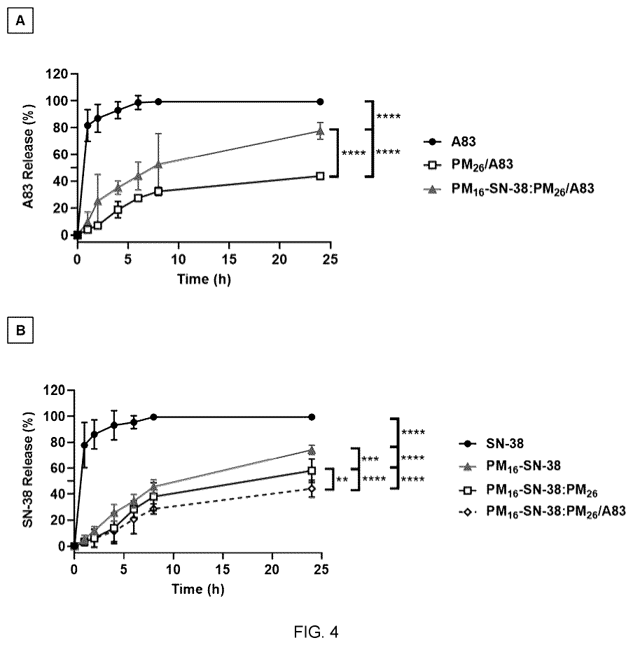

FIG. 4 shows the comparative in vitro release profiles of A83B4C63 and SN-38 from their respective formulations compared to free A83B4C63 and SN-38, under experimental conditions. Within 6 h, 98.81±2.09% of A83B4C63 and 96.46±1.43% of SN-38 as free drugs solubilize with the aid of DMSO were released from the dialysis bag indicating the existence of near sink condition [18]. In contrast, only 27.46±2.53% and 43.88±4.18% of A83B4C63 were released over 6 h from PM₂₆/A83 and PM₁₆-SN-38: PM₂₆/A83 micelles, respectively. Similarly, only 34.49±5.23%, 28.28±2.62%, and 20.70±4.52% of SN-38 were released over 6 h from PM₁₆-SN-38, PM₁₆-SN-38: PM₂₆, and PM₁₆-SN-38: PM₂₆/A83 micelles, respectively. In addition, the release of A83B4C63 from PM₁₆-SN-38: PM₂₆/A83 micelles over 24 h was 76.36±1.41% (FIG. 4A), which was significantly higher than that of PM₂₆/A83 (42.70±0.72) micelles and significantly lower that of free SN-38 (99.81±0.01%). In contrast, the release of SN-38 from PM₁₆-SN-38: PM₂₆/A83 micelles over 24 h was 44.15±2.61%, which was significantly lower than that of PM₁₆-SN-38 (74.16±3.65) and PM₁₆-SN-38: PM₂₆ micelles (57.94±3.69%) (FIG. 4B). The overall in vitro release study supports the efficiency of micellar formulations for sustaining the release of both A83B4C63 and SN-38. It also showed release of SN-38 to slow down but release of A83B4C63 to increase upon formation of mixed micelles (PM₁₆-SN-38: PM₂₆/A83) compared to individual micelles encapsulating each drug alone (PM₁₆-SN-38 or PM₂₆/A83).

### 3.5. Evaluation of Synergy Between the Combination Treatments

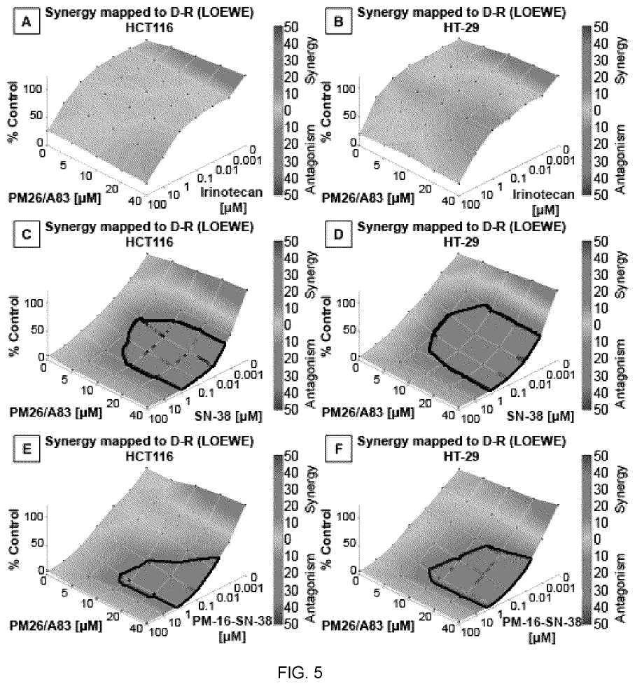

To further assess the synergistic versus additive effects of combinations of PNKP inhibitor and TOP1 inhibitor, and to predict the effective synergistic doses of these two drugs as combination therapy against CRC cell lines, we conducted software program-based analysis of our cell viability data. FIG. 5 shows the graphical outputs from the Combenefit® software that represent the combination dose response surface mapping the synergy as well as antagonism distribution. For interpreting the value of synergy scores, Combenefit® software has normalized input data as cell viability inhibition percentage to directly infer the proportion of cellular responses attributing to the interactions of various concentrations of both drugs. Therefore, the combination scores near zero in the surface plot yields inadequate confidence on synergy or antagonism. However, the combination scores below −10, between −10 to 10, and above 10 for the combination of two drugs are likely to be considered antagonism, additivity, and synergy, respectively. Irinotecan in combination with PNKP inhibitor mostly showed additive scores, also confirming the inefficacy to kill CRC cell lines as revealed by the cell viability experiments at studied concentrations (FIGS. 5A and 5B). Loewe models suggested the clear synergistic effects for combining encapsulated PNKP inhibitor and TOP1 inhibitor SN-38 (as free or encapsulated) at a concentration range between 0.01 to 10 μM for TOP1 inhibitor and between 10 to 40 μM for PNKP inhibitor. Since both SN-38 and PM₁₆-SN-38 are extremely toxic to cells, the combinatorial effects were not considered synergistic at higher concentrations of SN-38 by the software. When combining free SN-38 with PM₂₆/A83, the synergy scores reached to 50 in 10-40 μM range of A83B4C63 concentration (FIGS. 5C and 5D). Combining PM₂₆/A83 with PM₁₆-SN-38 produced a synergy score of 50 between 10 and 40 μM concentrations for A83B4C63 and 0.01 and 1 μM concentrations for SN-38 (FIGS. 5E and 5F).

### 3.6. In Vitro Cytotoxicity

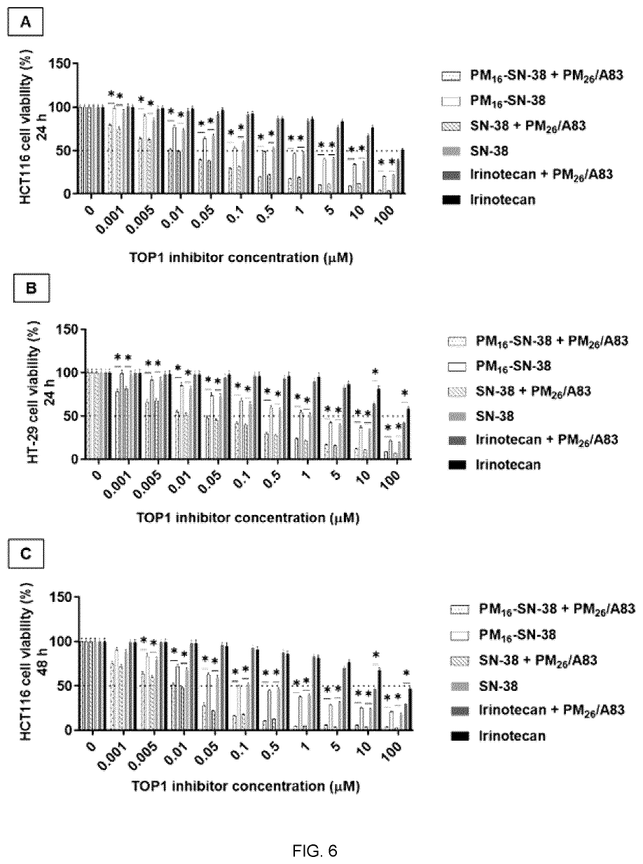

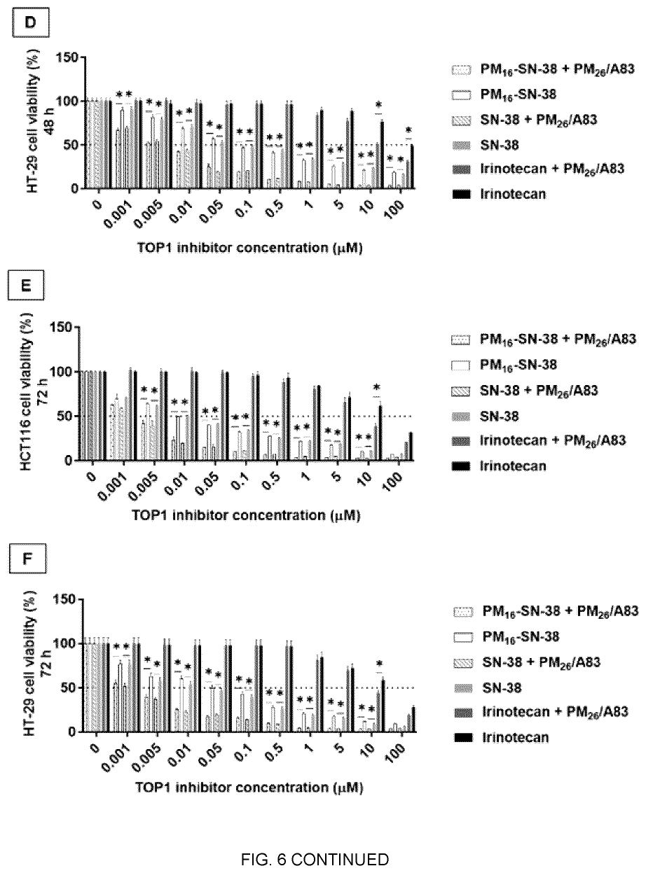

The MTS assay was performed at 24-72 h to determine the anticancer activity for the combination of TOP1 and PNKP inhibitors with or without nano-delivery against CRC cell lines (HCT116 and HT-29) in comparison to empty polymeric micelles, TOP1 inhibitors SN-38 or irinotecan alone, and PNKP inhibitor alone (FIG. 6).

As summarized in Table 3, in HCT116 the relative IC₅₀ of irinotecan was 7.3±3.7, 3.9±1.7 and 6.3±1.6 μM following 24, 48, and 72 h incubation. Upon combination of irinotecan with PM₂₆/A83 the relative IC₅₀ decreased to 5.7±2.5, 4.2+1.2, and 3.9±1.0 μM at the same incubation times, respectively. The difference in mono versus combination therapy was significant for the 72 h incubation time. Similar trend was observed in HT-29 cell line, where the decrease in relative IC₅₀ between mono and combination therapy was found to be significant after 48 h incubation time. As expected, at similar concentration range, treatment of both cell lines with SN-38 led to higher cytotoxicity compared to that for irinotecan (Table 3). In both cell lines, PM₁₆-SN-38 was shown to have similar relative IC₅₀ to that of free SN-38, in all incubation times.

Adding PM₂₆/A83 to both cell lines while treating them with either free SN-38 or PM₁₆-SN-38 led to a decrease in the relative IC₅₀ of SN-38 in all incubation times and in both cell lines. For instance, in HCT116 cells, the relative IC₅₀ of free SN-38 was decreased from 0.14±0.04, 0.05±0.01, 0.02±0.01 μM at 24, 48, and 72 h to 0.012+0.001, 0.007±0.001, and 0.002±0.001 μM upon combination with PM₂₆/A83. For PM₁₆-SN-38, co-treatment of HCT116 cells led to a decrease to relative IC₅₀ from 0.12±0.05, 0.05±0.01, and 0.01±0.002 μM to 0.013±0.001, 0.008±0.001, and 0.003±0.001 μM for 24, 48 and 72 h incubation times, respectively (*p<0.05, student's t-test). The results showed similarly superior cytotoxicity of SN-38+PM₂₆/A83 and that of PM₁₆-SN-38+PM₂₆/A83 samples over other treatments at all incubation time points against both cell lines.

TABLE 3
IC: range of irinotecan, irinotecan − [symbol] A83, SN-38, SN-38 − [symbol] A83, [symbol]
-SN-38, and [symbol] -SN-38 − [symbol] A83 against HCT116 and [symbol] -29 cell
lines after 24, 48, and 72 h of incubation (n = 4). [symbol] values were determined
after plotting the cell [symbol] percentages [symbol] various drug concentrations using
GraphPad Prism 9 software. The graph was then fitted with a non-linear regression
and sigmoid dose-response curve to obtain the [symbol] values. The number shown
in the subscript of the formulation names indicates the degree of polymerization
of each block of the copolymers as determined by H NMR spectroscopy.

| | | IC₅₀ (μM) | | | | | |
|---|---|---|---|---|---|---|---|
| | Time | | Irinotecan − | | SN-38 + | | PM₁₆-SN-38 + |
| Cells | (h) | Irinotecan | PM₂₆/A83 | SN-38 | PM₂₆/A83 | PM₁₆-SN-38 | PM₂₆/A83 |
| HCT116 | 24 | 7.3 [symbol] 3.7 | 5.7 [symbol] 2.5 | 0.14 [symbol] 0.04 | 0.012 [symbol] 0.001 | 0.12 [symbol] 0.05 | 0.013 [symbol] 0.001 |
| | 48 | 3.9 [symbol] 1.7 | 4.2 [symbol] 1.2 | 0.05 [symbol] 0.01 | 0.007 [symbol] 0.001 | 0.05 [symbol] 0.01 | 0.005 [symbol] 0.001 |
| | 72 | 6.3 [symbol] 1.6 | 3.9 [symbol] 1.0 | 0.02 [symbol] 0.01 | 0.002 [symbol] 0.001 | 0.01 [symbol] 0.002 | 0.003 [symbol] 0.001 |
| HT-29 | 24 | 10.7 ± 4.5 | 7.6 [symbol] 2.6 | 0.28 [symbol] 0.08 | 0.015 [symbol] 0.004 | 0.35 [symbol] 0.10 | 0.015 [symbol] 0.004 |
| | 48 | 11.6 ± 4.3 | 6.0 [symbol] 1.7 | 0.05 [symbol] 0.01 | 0.005 [symbol] 0.001 | 0.04 [symbol] 0.01 | 0.005 [symbol] 0.001 |
| | 72 | 7.1 [symbol] 2.2 | 5.8 [symbol] 1.7 | 0.02 [symbol] 0.01 | 0.002 [symbol] 0.001 | 0.02 [symbol] 0.01 | 0.002 [symbol] 0.001 |

[symbol]

indicates data missing or illegible when filed

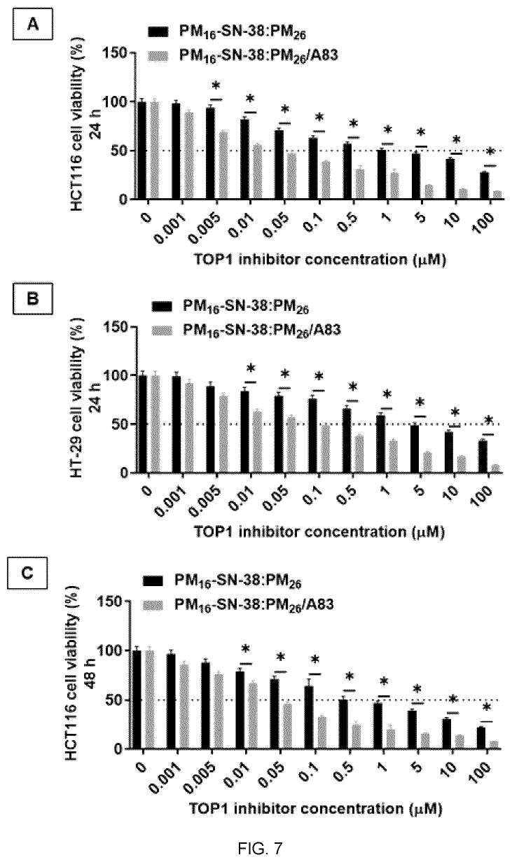

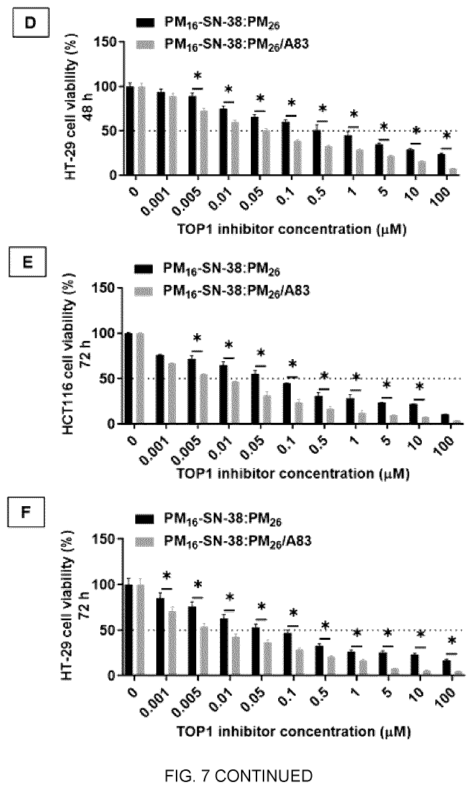

We then made a comparison between the cytotoxicity of PM₁₆-SN-38: PM₂₆ and that of PM₁₆-SN-38: PM₂₆/A83 co-delivery system (FIG. 7 and Table 4). Similar to combination treatment of PM₁₆-SN-38+PM₂₆/A83 that showed higher cytotoxicity in HCT116 and HT-29 cell lines compared to PM₁₆-SN-38, alone; the co-delivery system of PM₁₆-SN-38: PM₂₆/A83 was more cytotoxic than PM₁₆-SN-38: PM₂₆. In the HCT116 cells, the average relative IC₅₀ of co-delivery system was 0.02, 0.03 and 0.01 μM at 24, 48, and 72 h incubation, based on SN-38 concentration, while PM₁₆-SN-38: PM₂₆ without A83, showed relative IC₅₀s of 0.19, 0.16, and 0.03 μM at the same incubation times. This represents a 9.5, 5.3, and 3 fold decrease in the relative IC₅₀ of the co-delivery system over the same polymeric micellar structure without A83, which was similar to the level of reduction in relative IC₅₀ for the combination treatment of PM₁₆-SN-38+PM₂₆/A83 compared to PM₁₆-SN-38 alone. Similar trend was observed in HT-29 cells

TABLE 4
Relative IC₅₀ range of PM₁₆-SN-38:PM₂₆ and PM₁₆-SN-
38:PM₂₆/A83 against HCT116 and HT-29 cell lines after 24,
48, and 72 h of incubation (n = 4). The relative IC₅₀ values
were determined after plotting the cell viability percentages vs.
various drug concentrations using GraphPad Prism 9 software. The
graph was then fitted with a non-linear regression and sigmoid dose-
response curve to obtain the relative IC₅₀ values. The number
shown in the subscript of the formulation names indicates the
degree of polymerization of each block of the copolymers as
determined by ¹H NMR spectroscopy

| | Time | IC₅₀ (μM) | |
|---|---|---|---|
| Cells | (h) | PM₁₆-SN-38:PM₂₆ | PM₁₆-SN-38:PM₂₆/A83 |
| HCT116 | 24 | 0.19 ± 0.07 | 0.02 = 0.007 |
| | 48 | 0.16 ± 0.05 | 0.03 = 0.005 |
| | 72 | 0.03 ± 0.01 | 0.01 = 0.001 |
| HT-29 | 24 | 0.32 ± 0.11 | 0.07 = 0.02 |
| | 48 | 0.10 ± 0.03 | 0.04 = 0.01 |
| | 72 | 0.03 ± 0.01 | 0.01 = 0.001 |

### 3.7. Expression of Apoptosis Mediators for the Combination Versus Monotherapies

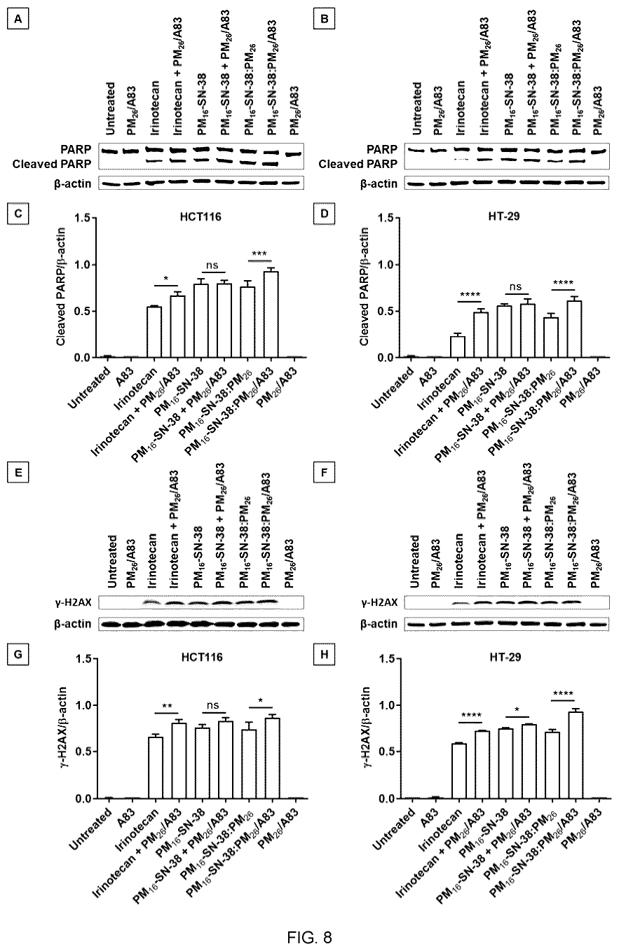

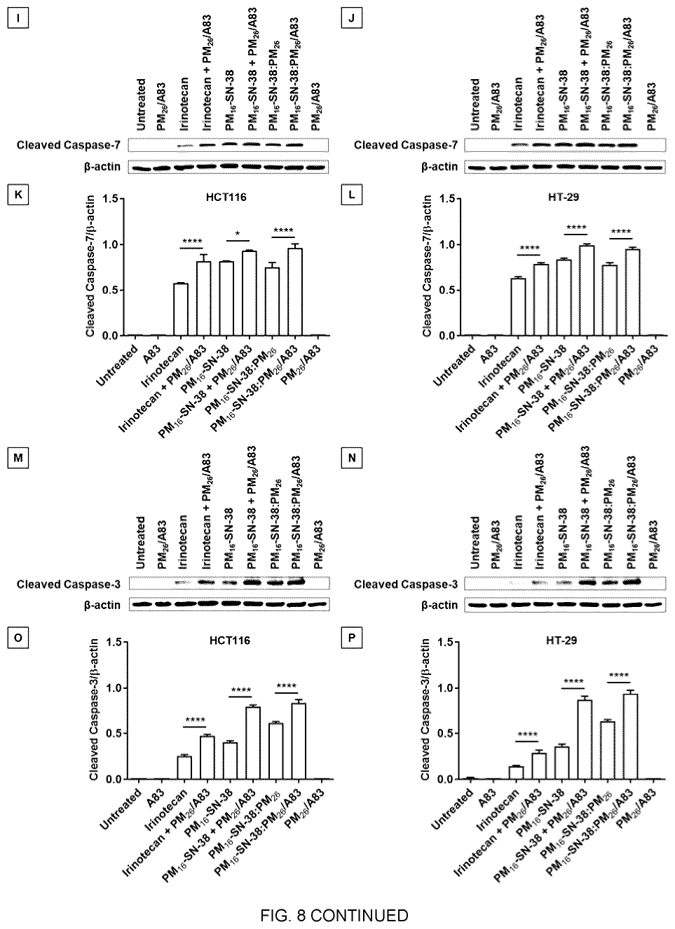

As shown in FIG. 8, PM₂₆, free A83 and PM₂₆/A83 showed no apoptotic signaling reflecting the inertness and nontoxic characteristics of the delivery system and lack of off target activity by the PNKP inhibitor A83B4C63 at 10 μM. Addition of PM₂₆/A83 as PNKP inhibitor to irinotecan significantly increased the expression of cleaved PARP, g-H2AX, cleaved caspase 3 and 7 in both cell lines. Addition of PM₂₆/A83 to PM₁₆-SN-38, did not affect cleaved PARP, g-H2AX expression significantly except in HT-29 cells where g-H2AX expression was increased with the co-treatment. The cleaved caspase 3 expression, however showed a significant increase in expression in both cell lines upon co-treatment with these formulations compared to monotherapy with PM₁₆-SN-38, however (*p<0.05).

In HCT116 cells, the formulation designed for co-delivery of TOP1 and PNKP inhibitor, i.e., PM₁₆-SN-38: PM₂₆/A83 at its relative IC₅₀ concentration revealed significantly higher expression of cleaved PARP (*p≤0.05, student's t-test), when compared to the co-treatment with individual formulations, i.e., PM₂₆/A83+PM₁₆-SN-38. However, no significant difference was observed for the expression of cleaved PARP between these treatments in HT-29 cell line. Moreover, in HT-29 cells, the PM₁₆-SN-38: PM₂₆/A83 treatment showed significantly higher expression of g-H2AX (*p≤0.05, student's t-test) compared to the co-treatment with the individual formulations, i.e., PM₂₆/A83+PM₁₆-SN-38. In contrast, no significant difference was observed for the expression of g-H2AX between these treatments in HCT116 cell line. Notably, no significant differences were obtained between co-delivery formulation (PM₁₆-SN-38: PM₂₆/A83) and combination (PM₁₆-SN-38+PM₂₆/A83) treatments in both cell lines in the expression cleaved caspase 3 and 7. Based on these data, the similar expression levels of these apoptosis mediators in CRC cell lines upon co-delivery (PM₁₆-SN-38: PM₂₆/A83) and combination (PM₁₆-SN-38+PM₂₆/A83) treatments demonstrated both structural integrity and anticancer efficacy of the payloads using polymeric micellar NPs.

### 4. DISCUSSION

The resistances to the conventional chemotherapies and the development of metastasis are the main causes of poor prognosis for advanced CRC. In last 2-3 decades, CRC patients have been exclusively treated with the chemotherapies based on fluoropyrimidines until the recognition of TOP1 enzyme as a valid therapeutic target in the cancer. As a result, a second generation semisynthetic camptothecin derivative, i.e., irinotecan as TOP1 inhibitor has been approved for clinical use in CRC patients. However, irinotecan is a prodrug which needs enzymatic conversion to produce its active metabolite, SN-38 in liver. Such conversion in liver needs higher frequent doses of irinotecan and eventually it results in suboptimal therapeutic responses, which requires combination treatment(s) in most advanced and some primary CRC patients [19].

Our research group has previously reported on the development of novel inhibitors of a DNA repair enzyme known as PNKP that can sensitize CRC to the cytotoxic effect of TOP1 inhibitors. We have also developed polymeric micellar formulations for SN-38 as well as a lead PNKP inhibitor known as A83B4C63 [2, 11, 20]. The primary objective of this study was to investigate the synergy between polymeric micellar formulations of SN-38 and A83B4C63 combination as individual micelles added together or mixed micelles co-delivering both drugs (Table 1) against CRC cell lines, in vitro.

First, SN-38 was chemically conjugated to the poly(ester) end of mPEO-b-PBCL with a lower DP of 16, using activation of the COOH group at the polymer end. This has led to >600 folds increase in water solubilized levels of SN-38 (25 μg/mL to over 15 mg/mL). The level of SN-38 conjugation to the carboxyl group at the PBCL end increased by ˜5% where activation of carboxyl group by oxalyl chloride was pursued, compared to our previously reported SN-38 conjugation [11]. For comparison, the conjugated level of SN-38 to PEO-poly(glutamic acid) polymers in the NK012 formulation is reported at 20% w/w, while SN-38 conjugation to the mPEO-b-PBCL end of this paper is at 16% w/w [21].

With respect to A83B4C63 encapsulation, the use of mixed block copolymers of PM₁₆-SN-38: PM₂₆ instead of PM₂₆ alone led to a decrease in the encapsulated levels of A83B4C63 (Table 2). Although the achieved loading still increased the water-soluble levels of A83B4C63, from 1 μM to 10.67.

Micelles co-encapsulating SN-38 and A83, showed increased average diameter compared to polymeric micelles encapsulating each drug, although the average diameter of the co-delivery systems. was still <60 nm (Table 2 and FIG. 2). Like polymeric micelles delivering individual SN-38 or A83, the mixed micelles containing both drugs showed high thermodynamic and kinetic stability (Table 2 and FIG. 3). The high stability of the polymeric micellar structure is mainly attributed to the presence of benzyl carboxylate group that may lead to p-p stacking and rigidity of the core structure [11]. Similar to micelles for individual drugs, the releases of SN-38 and A83B4C63 from the mixed micellar formulation co-encapsulating both drugs was sustained compared to free drugs [11]. However, the release of A83B4C63 was higher from the co-delivery systems compared to micelles encapsulating A83B4C63 alone, the release of SN-38 was lower from the co-delivery system compared to that of PM-SN-38 alone.

We then investigated the synergy between SN-38 as free and polymeric micellar formulation with that of PM₂₆/A83 formulation at different concentrations and ratios, first, using Combenefit® software. Our data revealed a synergy between free SN-38 and PM₁₆-SN-38 with PM₂₆/A83 at a concentration range of 0.01-1 μM for SN-38 and 10-40 μM for A83 (FIG. 5).

Thereafter, we assessed the efficacy for this combination formulation in human CRC cell lines while keeping the concentration of A83 fixed at 10 μM. The cytotoxicity data revealed significantly higher cell viability reduction for the combination treatments compared to their counterparts with SN-38 treatment only. Perhaps due to the efficient intracellular release of SN-38, all micellar SN-38 formulations showed significantly higher cell viability reduction than conventional irinotecan with or without PNKP inhibitor in human CRC cell lines. The effect of combination treatment with PNKP+TOP1 inhibitor was also observed in the increased DNA damage and mediators of apoptosis in CRC cells compared to their respective individual treatments with TOP1 inhibitor within 6 h.

In both HCT116 and HT-29 CRC cell lines, the co-delivery of TOP1 and PNKP inhibitors using mixed micelles induced significant cytotoxicity, DNA damage and increase of apoptosis biomarkers compared to its counterpart with TOP1 inhibitor encapsulation only [4], while the PNKP enzyme involved in repairing the single strand DNA breaks by SN-38 similar DNA damaging agents. Ideally, PNKP catalyzes the restoration of 5′-phosphate and 3′-hydroxyl termini and allow DNA ligase to allow the rejoin the breaks. PNKP also ensures that 5′-OH termini are phosphorylated although its phosphatase activity was predominant over the kinase activity in strand breaks with both 3′-phosphate and 5′-OH termini. When TOP1-DNA “dead-end” complex is generated by SN-38, TOP1 enzyme was found to be attached to the DNA 3′-phosphate covalently through a tyrosine residue and subsequently released from the DNA by Tyrosyl-DNA Phosphodiesterase 1 or TDP1 for processing prior to ligation steps. Therefore, PNKP was found to participate in the correction 3′-phosphate and 5′-OH termini of the DNA breaks [22]. In case of regular single stranded DNA breaks, PNKP is not considered to be the underlying DNA repair enzyme unless TOP1 is inhibited by TOP1 inhibitor. Similarly in this study, the non-toxicity of both free and micellar PNKP inhibitors were evidenced by no cell viability reduction up to 40 μM dose after 72 h treatment and also further verified by no expression levels of cleaved PARP and γ-H2AX in CRC cell lines. Due to non-toxic behavior of our novel PNKP inhibitor, relative IC₅₀ concentration was not measurable up to 40 μM treating dose which was 3 times higher treating dose than the required dose (10 μM) for desired chemo-sensitization in this study. This observation was also in agreement with our previous in vivo study where neither systemic nor tissue toxicity was identified in healthy CD-1 mice receiving A83B4C63 up to 50 mg/kg dose intravenously either solubilized with the aid of Cremophor EL: Ethanol formulation or similar micellar formulation (PM₂₆/A83) investigated in this study [20].

It appears that the co-delivery of A83B4C63 and SN-38 in one polymeric micellar formulation (PM₁₆-SN-38: PM₂₆/A83), potentiated the effect of A83B4C63. This was evident from similar cell viability at much lower ratios of A83 to that of SN-38 in co-delivery systems (PM₁₆-SN-38: PM₂₆/A83) in both cell lines compared to that of combination treatment (PM₁₆-SN-38+PM₂₆/A83).

### 5. CONCLUSION

Most importantly, the chemical conjugation of SN-38 to the hydrophobic core of self-associating PEO-poly(ester) micelles was successful with a higher w/w loading in this study, leading to a suitable water-soluble SN-38 formulation for systemic administration. Co-delivery of both TOP1 and PNKP inhibitors in PEO-poly(ester) based nano-carriers showed promising in vitro chemo-sensitizing synergy in human CRC cell lines. The overall findings demonstrate PM₁₆-SN-38: PM₂₆ as a co-delivery platform for a novel combination treatment option against CRC.

### ABBREVIATIONS

BCL, α-benzyl carboxylate-ε-caprolactone; BSA, bovine serum albumin; CDCl₃, deuterated chloroform; CMC, critical micellar concentration; CRC, colorectal cancer; DIC, N,N′-diisopropylcarbodiimide; DLS, dynamic light scattering; DMAP, 4-dimethylaminopyridine; DMEM/F12, dulbecco's modified eagle medium and F12; DMF, dimethylformamide; DMSO, dimethyl sulfoxide; DNA, deoxy ribonucleic acid; DP, degree of polymerization; EPR, enhanced permeability and retention effect; Kcps, kilo counts per second; mPEO, methoxy-polyethylene oxide; mPEO-b-PBCL, methoxy-poly(ethylene oxide)-b-poly(α-benzyl carboxylate-ε-caprolactone); mPEO-b-PBCL/SN-38, SN-38-incorporated mPEO-b-PBCL micelle; mPEO-b-PCCL, methoxy-poly(ethylene oxide)-b-poly(α-carboxyl-8-caprolactone); mPEO-b-PCCL/SN-38, SN-38-incorporated mPEO-b-PCCL micelle; MW, molecular weight; PDI, polydispersity index; RBC, red blood cell; SDS, sodium dodecyl sulfate; SN-38, 7-ethyl-10-hydroxy-camptothecin; TEM, transmission electron microscopy; THF, tetrahydrofuran; TOP1, topoisomerase I; ZP, zeta potential.

### REFERENCES

1. Alfayez, M., et al., CPX-351 (vyxeos) in AML. Leuk Lymphoma, 2020. 61 (2): p. 288-297.
2. Shire, Z., et al., Nanoencapsulation of Novel Inhibitors of PNKP for Selective Sensitization to Lonizing Radiation and Irinotecan and Induction of Synthetic Lethality. Mol Pharm, 2018. 15 (6): p. 2316-2326.
3. Freschauf, G. K., et al., Identification of a small molecule inhibitor of the human DNA repair enzyme polynucleotide kinase/phosphatase. Cancer Res, 2009. 69 (19): p. 7739-46.
4. Palakurthi, S., Challenges in SN38 drug delivery: current success and future directions. Expert Opin Drug Deliv, 2015. 12 (12): p. 1911-21.
5. Goldwirt, L., et al., A new UPLC-MS/MS method for the determination of irinotecan and 7-ethyl-10-hydroxycamptothecin (SN-38) in mice: application to plasma and brain pharmacokinetics. J Pharm Biomed Anal, 2012. 66: p. 325-33.
6. Doi, H., et al., Solubility-Improved 10-O-Substituted SN-38 Derivatives with Antitumor Activity. ChemMedChem, 2017.
7. Garcia-Carbonero, R. and J. G. Supko, Current perspectives on the clinical experience, pharmacology, and continued development of the camptothecins. Clin Cancer Res, 2002. 8 (3): p. 641-61.
8. Wang, W., et al., Effective conversion of irinotecan to SN-38 after intratumoral drug delivery to an intracranial murine glioma model in vivo. Laboratory investigation. J Neurosurg, 2011. 114 (3): p. 689-94.
9. Mahmud, A., X. B. Xiong, and A. Lavasanifar, Development of novel polymeric micellar drug conjugates and nano-containers with hydrolyzable core structure for doxorubicin delivery. Eur J Pharm Biopharm, 2008. 69 (3): p. 923-34.
10. Toure, B. B., et al., A three-component reaction for diversity-oriented synthesis of polysubstituted piperidines: solution and solid-phase optimization of the first tandem aza [4+2]/allylboration. Chemistry, 2003. 9 (2): p. 466-74.
11. Sadat, S. M. A., et al., Development of Self-Associating SN-38-Conjugated Poly(ethylene oxide)-Poly(ester) Micelles for Colorectal Cancer Therapy. Pharmaceutics, 2020. 12 (11): p. 1033.
12. Hakala, R. A., et al., Hydrophobicities of poly(ε-caprolactone) oligomers functionalized with different succinic anhydrides. European Polymer Journal, 2009. 45 (2): p. 557-564.
13. Garg, S. M., M. R. Vakili, and A. Lavasanifar, Polymeric micelles based on poly(ethylene oxide) and alpha-carbon substituted poly(varepsilon-caprolactone): An in vitro study on the effect of core forming block on polymeric micellar stability, biocompatibility, and immunogenicity. Colloids Surf B Biointerfaces, 2015. 132: p. 161-70.
14. Saqr, A., et al., Development of Traceable Rituximab-Modified PEO-Polyester Micelles by Postinsertion of PEG-phospholipids for Targeting of B-cell Lymphoma. ACS Omega, 2019. 4 (20): p. 18867-18879.
15. Di Veroli, G. Y., et al., Combenefit: an interactive platform for the analysis and visualization of drug combinations. Bioinformatics, 2016. 32 (18): p. 2866-8.
16. Yu, S., et al., Total synthesis of camptothecin and SN-38. J Org Chem, 2012. 77 (1): p. 713-7.
17. Liu, Y., et al., Comparison of two self-assembled macromolecular prodrug micelles with different conjugate positions of SN38 for enhancing antitumor activity. Int J Nanomedicine, 2015. 10: p. 2295-311.
18. Aliabadi, H. M. and A. Lavasanifar, Polymeric micelles for drug delivery. Expert Opin Drug Deliv, 2006. 3 (1): p. 139-62.
19. Xie, Y.-H., Y.-X. Chen, and J.-Y. Fang, Comprehensive review of targeted therapy for colorectal cancer. Signal Transduction and Targeted Therapy, 2020. 5 (1): p. 22.
20. Sadat, S. M. A., et al., A synthetically lethal nanomedicine delivering novel inhibitors of polynucleotide kinase 3′-phosphatase (PNKP) for targeted therapy of PTEN-deficient colorectal cancer. J Control Release, 2021. 334: p. 335-352.
21. Koizumi, F., et al., Novel SN-38-incorporating polymeric micelles, NK012, eradicate vascular endothelial growth factor-secreting bulky tumors. Cancer Res, 2006. 66 (20): p. 10048-56.
22. Weinfeld, M., et al., Tidying up loose ends: the role of polynucleotide kinase/phosphatase in DNA strand break repair. Trends in biochemical sciences, 2011. 36 (5): p. 262-271.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of treating a subject having colorectal cancer, suspected of having of treating colorectal cancer, or at risk of developing of treating colorectal cancer, comprising:
administering to said subject,
a therapeutically effective amount of A83B4C63, or a pharmaceutically acceptable salt, solvate, hydrate, or prodrug thereof, and
a therapeutically effective amount of SN-38, or a pharmaceutically acceptable salt, solvate, hydrate, or prodrug thereof.

2. The method of claim 1, wherein the SN-38, or a pharmaceutically acceptable salt, solvate, hydrate, or prodrug thereof is administered before administration of the A83B4C63, or a pharmaceutically acceptable salt, solvate, hydrate, or prodrug thereof.

3. The method of claim 1 or 2, wherein the A83B4C63, or a pharmaceutically acceptable salt, solvate, hydrate, or prodrug thereof is administered before administration of the PM₁₆-SN-38, or a pharmaceutically acceptable salt, solvate, hydrate, or prodrug thereof.

4. The method of claim 1, wherein the SN-38, or a pharmaceutically acceptable salt, solvate, hydrate, or prodrug thereof is administered substantially concurrently with the A83B4C63, or a pharmaceutically acceptable salt, solvate, hydrate, or prodrug thereof.

5. The method of any one of claims 1 to 4, wherein the subject is a human.

6. The method of any one of claims 1 to 5, wherein the therapeutically effective amount of A83B4C63 is administered in a micelle.

7. The method of claim 6, wherein the A83B4C63-containing micelle comprises block copolymer mPEO-b-PBCL, PEO-b-PBCL, PEO-PCL, PEO-PDLA, and/or PEO-PLGA.

8. The method of claim 7, wherein the A83B4C63-containing micelle comprises block copolymer mPEO-b-PBCL.

9. The method of claim 8, wherein the PBCL block of the block copolymer in the A83B4C63-containing micelle has a degree of polymerization of between 2 and 50, preferably 10-30.

10. The method of claim 9, wherein the PBCL block of the block copolymer in the A83B4C63-containing micelle has a degree of polymerization of 26.

11. The method of claims 6-10, wherein the concentration of A83B4C63 in the A83-containing micelle is between 1 μM and 100 μM, preferably between 5 μM and 40 μM.

12. The methods of any one of claims 1 to 11, wherein the therapeutically effective amount of SN-38 is administered in a micelle.

13. The method of claim 12, wherein the SN-38-containing micelle comprises block copolymer mPEO-b-PBCL, PEO-b-PBCL, PEO-PCL, PEO-PDLA, and/or PEO-PLGA.

14. The method of claim 13, wherein the SN-38-containing micelle comprises block copolymer mPEO-B-PBCL.

15. The method of claims 13 to 14, wherein SN-38 in the SN-38-containing micelle is conjugated to a block copolymer.

16. The method of claims 13 to 15, wherein the PBCL block of the block copolymer in the SN-38-containing micelle has a degree of polymerization of between 1 and 30, preferably between 5 and 25.

17. The method of claims 13 to 16, wherein the PBCL block of the block copolymer in the SN-38-containing micelle has a degree of polymerization of 16.

18. The method of claims 12 to 17, wherein the concentration of SN-38 in the micelle is between 0.001 μM and 10 μM, preferably between 0.005 μM and 1 μM.

19. The method of claims 1 to 5, wherein the therapeutically effective amount of A83B4C63 or SN-38 is administered in a composition.

20. The method of claims 1 to 5, wherein the therapeutically effective amount of A83B4C63 or SN-38 is administered in a pharmaceutical composition further comprising a pharmaceutically acceptable excipient, diluent, or carrier.

21. Use of a therapeutically effective amount of A83B4C63, or a pharmaceutically acceptable salt, solvate, hydrate, or prodrug thereof, and a therapeutically effective amount of SN-38, or a pharmaceutically acceptable salt, solvate, hydrate, or prodrug thereof, for treating a subject having colorectal cancer, suspected of having of treating colorectal cancer, or at risk of developing of treating colorectal cancer.

22. Use of a therapeutically effective amount of A83B4C63, or a pharmaceutically acceptable salt, solvate, hydrate, or prodrug thereof, and a therapeutically effective amount of SN-38, or a pharmaceutically acceptable salt, solvate, hydrate, or prodrug thereof, in the manufacture of a medicament for treating a subject having colorectal cancer, suspected of having of treating colorectal cancer, or at risk of developing of treating colorectal cancer.

23. The use of claim 21 or 22, wherein the SN-38, or a pharmaceutically acceptable salt, solvate, hydrate, or prodrug thereof is for administration before administration of the A83B4C63, or a pharmaceutically acceptable salt, solvate, hydrate, or prodrug thereof.

24. The use of claim 21 or 22, wherein the A83B4C63, or a pharmaceutically acceptable salt, solvate, hydrate, or prodrug thereof is for administration before administration of the SN-38, or a pharmaceutically acceptable salt, solvate, hydrate, or prodrug thereof.

25. The use of claim 21 or 22, wherein the SN-38, or a pharmaceutically acceptable salt, solvate, hydrate, or prodrug thereof is for administration substantially concurrently with the A83, or a pharmaceutically acceptable salt, solvate, hydrate, or prodrug thereof.

26. The use of any one of claims 18-25, wherein the subject is a human.

27. A micelle composition comprising SN-38 and A83B4C63.

28. The micelle composition of claim 24, wherein the micelle comprises block copolymer mPEO-b-PBCL, PEO-b-PBCL, PEO-PCL, PEO-PDLA, and/or PEO-PLGA.

29. The micelle composition of claim 28, wherein the micelle comprises block copolymer mPEO-b-PBCL.

30. The micelle composition of claims 27 to 29, wherein SN-38 is conjugated to a block copolymer.

31. The micelle composition of claims 27 to 30, wherein the PBCL block of the block copolymer has a degree of polymerization between 1 and 50.

32. The micelle composition of claims 27 to 31, wherein the concentration of A83B4C63 is between 1 μM and 100 μM, preferably between 5 μM and 40 μM, and the concentration of SN-38 is between is between 0.001 μM and 10 μM, preferably between 0.005 μM and 1 μM.

33. A composition comprising SN-38 and A83B4C63.

34. A pharmaceutical composition comprising SN-38 and A83B4C63, a pharmaceutically acceptable excipient, diluent, or carrier.

35. A method of treating colorectal cancer in a subject with of treating colorectal cancer, suspected of having of treating colorectal cancer, or at risk of developing of treating colorectal cancer, comprising: administering to said subject a therapeutically effective amount a micelle composition of claims 27 to 32, a composition of claim 33, or a pharmaceutical composition of claim 34.

36. The method of claim 35, wherein the subject is a human.

37. Use of a therapeutically effective amount of a micelle composition of claims 27 to 32, a composition of claim 33, or a pharmaceutical composition of claim 34, or a pharmaceutically acceptable salt, solvate, hydrate, or prodrug thereof, for treating a subject having colorectal cancer, suspected of having of treating colorectal cancer, or at risk of developing of treating colorectal cancer.

38. Use of a therapeutically effective amount of a micelle composition of claims 27 to 32, a composition of claim 33, or a pharmaceutical composition of claim 34, or a pharmaceutically acceptable salt, solvate, hydrate, or prodrug thereof, in the manufacture of a medicament for treating a subject having colorectal cancer, suspected of having of treating colorectal cancer, or at risk of developing of treating colorectal cancer.

39. The use of claim 37 or 38, wherein the subject is a human.
